(19) United States
(12) Patent Application Publication
(10) Pub. No.: US 2012/0270915 A1
(43) Pub. Date: Oct. 25, 2012

(54) FATTY ACID AMIDE HYDROLASE INHIHIBITORS FOR TREATING PAIN

(76) Inventors: David F. Woodward, Lake Forest, CA (US); Jose L. Martos, Basildon Essex (GB); William R. Carling, Bishop's Stortford (GB); Neil J. Poloso, Lake Forest, CA (US); Jenny W. Wang, Irvine, CA (US)

(21) Appl. No.: 13/453,068

(22) Filed: Apr. 23, 2012

Related U.S. Application Data
(60) Provisional application No. 61/478,225, filed on Apr. 22, 2011.

(51) Int. Cl. A61K 31/422; A61P 25/00; C07D 413/04

(52) U.S. Cl. 514/374; 548/238

(57) ABSTRACT

Compounds of Formula 1 are described herein. These compounds may be administered to a patient for treatment of suffering from pain or other FAAH mediated conditions.

### CROSS-REFERENCE TO RELATED APPLICATIONS

This application claims the benefit of U.S. Provisional Patent Application 61/478,225, filed Apr. 22, 2011, which is incorporated by reference herein in its entirety.

Embodiments described herein relate to a method for treating pain and other diseases and conditions of the central nervous system (CNS) and peripheral nervous system (PNS) by inhibiting the action of fatty acid amide hydrolase in the body of a patient in need of treatment therefore to thereby modulate the breakdown of naturally occurring endocannabinoids, such as anandamide. In addition, blockade of prostanoid receptors provides additional benefit.

### BACKGROUND

Fatty acid amide hydrolase (FAAH) is an enzyme that modulates central nervous system (CNS) functions such as pain perception, cognition, feeding, sleep and locomotion by breaking down certain fatty signaling molecules that reside in the lipid membranes of CNS cells

The structure of this enzyme was described in the journal, Science, by researchers from the Scripps Institute. The Scripps researchers reported that FAAH modulates the action of these fatty signaling molecules through an unusual mechanism whereby it “scoops” such molecules out of the cell membranes and “chews” them up.

The researchers surmised that the deep pocket with well-defined cavities provided the guidance to take the currently available tight binding inhibitors and improve on their specificity and pharmacokinetic properties.

The researchers also surmised that a specific inhibitor to FAAH could, in principal, provide pain relief without any side effects.

There is an ongoing search for compounds that not only ease pain, but do so as fast, effectively, and as lastingly as possible—and without any unwanted side effects; however every analgesic, from opiates to hypnotism to electroshocks to balms, have side effects.

Delta-9-tetrahydrocannabinol (THC), the active ingredient in marijuana, works as an analgesic by mimicking the action of natural mammalian endocannabinoids that the body produces in signaling cascades in response to a peripheral pain stimulus. THC binds to “CB-1” receptors on cells on the rostral ventromedial medulla, a pain-modulating center of the brain, decreasing sensitivity to pain.

However, the receptors that THC binds to are also widely expressed in other parts of the brain, such as in the memory and information-processing centers of the hippocampus. Binding to nerve cells of the hippocampus and other cells elsewhere in the body, THC creates a range of side effects as it activates CB-1 mediated signaling—including distorted perception, difficulty in problem-solving, loss of coordination, and increased heart rate and blood pressure, anxiety and panic attacks.

The challenge thus posed by THC and other cannabinoids is to find a way to use them to produce effective, long-lasting relief from pain without the deleterious side effects.

It has been suggested that the solution is to increase the efficacy of the natural, endogenous cannabinoids (“endocannabinoids”) the body produces to modulate pain sensations.

The amplitude and duration of the activity of such endocannabinoids are regulated by how fast they are broken down.

In particular, the body releases an endogenous cannabinoid called anandamide. When the body senses pain, anandamide binds to CB-1 and nullifies pain by blocking the signaling. However, this effect is weak and short-lived as FAAH quickly metabolizes anandamide, as the compound has a half-life of only a few minutes in vivo.

In some ways, THC is superior to anandamide as a pain reliever because it is not as readily metabolized by FAAH. But, since THC goes on to interact with cannabinoid receptors all over the body and it is a controlled substance, THC is an unattractive target for developing therapeutics, as compared to FAAH.

FAAH is a much more attractive target for pain therapy because by inhibiting FAAH, you would increase the longevity of anandamide molecules—preventing their breakdown and allowing them to continue providing some natural pain relief.

Thus, designing specific inhibitors that control the action of FAAH when the body is sensing pain and releasing anandamide is very desirable.

### SUMMARY

Some embodiments include a compound represented by Formula 1:

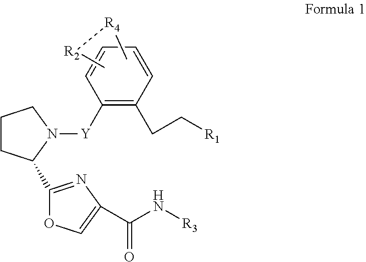

wherein a dashed line indicates the presence or absence of a bond; R₁ is an acyl sulfonamide moiety or CO₂H; R₂ and R₄ are independently H, alkyl, halo or alkyloxy; R₃ is H or alkyl; and Y is CO or (CH₂)ₙ, wherein n is 1, 2, or 3.

Methods for inhibiting the activity of fatty acid amide hydrolase (FAAH) and multiple prostanoid receptors in a human to thereby modulate central nervous system (CNS) functions such as pain perception, cognition, feeding, sleep, and locomotive activity are also described herein. Some methods function to attenuate the break down of certain fatty signaling molecules that reside in the lipid membranes of CNS cells by treating a patient in need of the treatment with an effective amount of a compound described herein, such as a compound of Formula 1 or another formula herein (referred to collectively as “the compounds”).

### DETAILED DESCRIPTION

Unless otherwise stated the following terms used in the specification and claims have the meanings discussed below:

“Hydrocarbyl” includes a hydrocarbon moiety having only carbon and hydrogen atoms. In some embodiments, the hydrocarbyl moiety has from 1 to 20 carbon atoms, from 1 to 12 carbon atoms, or from 1 to 7 carbon atoms.

“Substituted hydrocarbyl” includes a hydrocarbyl moiety wherein one or more, but not all, of the hydrogen and/or the carbon atoms are replaced by one or more halogen, nitrogen, oxygen, sulfur or phosphorus atoms or a moiety including a halo, nitrogen, oxygen, sulfur or phosphorus atom, e.g. fluoro, chloro, cyano, nitro, dialkylamino, hydroxyl, phosphate, thiol, etc.

“Alkyl” includes a straight-chain, branched or cyclic saturated aliphatic hydrocarbon. In some embodiments, the alkyl group has 1 to 20 carbons, 1 to 12 carbons, or 1 to 10 carbons. Typical alkyl groups include methyl, ethyl, n-propyl, isopropyl, butyl, isobutyl, tertiary butyl, pentyl, hexyl and the like as well as cycloalkyl-n-alkyl groups such as cyclohexyl-n-butyl. The alkyl group may be optionally substituted with one or more substituents such as hydroxyl, cyano, alkoxy, ═O, ═S, NO₂, halo, dimethyl amino, and SH. Haloalkyl includes alkyl having one or more halogen substituents, such as fluoroalkyl (e.g. CF₃, CH₂CH₂CH₂F, etc.)

“Cycloalkyl” includes a cyclic saturated aliphatic hydrocarbon group. In some embodiments, the cycloalkyl group has 3 to 12 carbons, 4 to 7 carbons, or 5 or 6 carbons.

“Aryl” includes an aromatic group such as carbocyclic aryl, heterocyclic aryl and biaryl groups. An aryl group may be optionally substituted with one or more substituents such as alkyl, hydroxyl, halo, COOR₆, NO₂, CF₃, N(R₆)₂, CON(R₆)₂, SR₆, sulfoxy, sulfone, CN and OR₆, wherein R₆ is alkyl.

“Carbocyclic aryl” includes an aryl group wherein the ring atoms are carbon.

“Heteroaryl” or “heterocyclic aryl” includes a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, purine, tetrazole, triazine, and carbazole. The heteroaryl group may be substituted or unsubstituted.

“Hydroxyl” refers to an —OH group.

“Alkoxy” refers to an —O-(alkyl) an —O-(cycloalkyl) or an —O-alkyl-O— group. Representative examples include, but are not limited to, e.g., methoxy, ethoxy, propoxy, butoxy, dioxol, cyclopropyloxy, cyclobutyloxy, cyclopentyloxy, cyclohexyloxy, and the like.

“Acyl” refers to a —C(O)— group

“Halo” refers to fluorine, chlorine, bromine or iodine, preferably fluorine or chlorine.

“Dialkylamino” includes a moiety —NRR where each R is independently an alkyl or cycloalkyl group as described above, e.g., dimethylamino, diethylamino, (1-methylethyl)-ethylamino, cyclohexylmethylamino, cyclopentylmethylamino, and the like.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

Any structure or name for a compound used herein may refer to any stereoisomer of the compound or any mixture of stereoisomers including the compound.

The compounds may be represented by Formula 1 above, or any of Formulas 2-7 below:

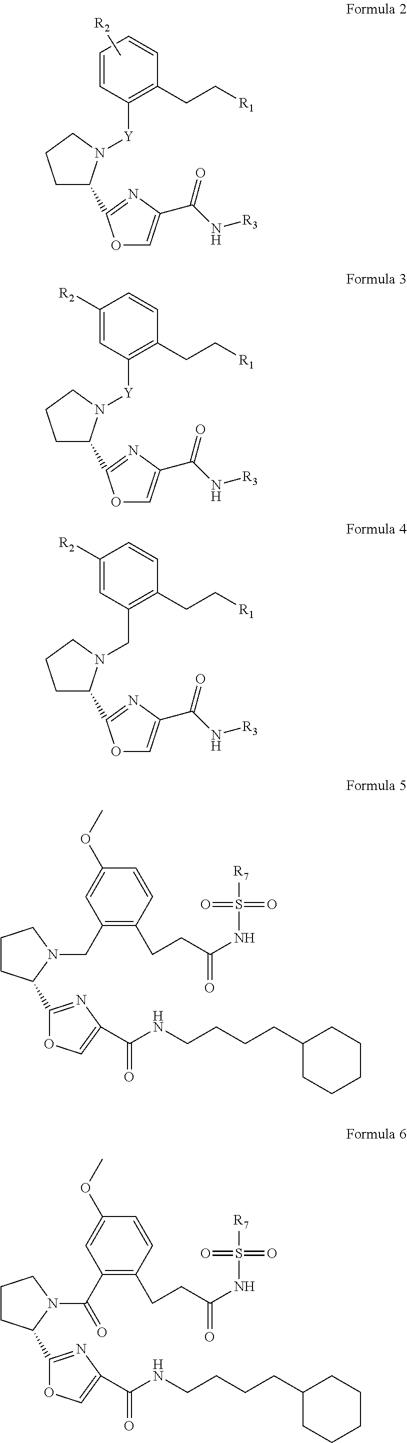

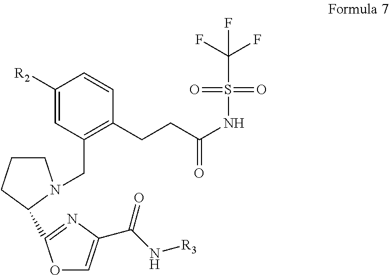

wherein R₁, R₂, R₃, R₄, and Y are as defined above.

In some embodiments, Y is CO or CH₂

In some embodiments, R₁ is CO₂H, CON(R₇)SO₂R₇ or CON(H)SO₂R₇.

R₇ may be H, substituted or unsubstituted hydrocarbyl, substituted or unsubstituted aryl, or dialkylamino. In some embodiments, R₇ may be alkyl, dialkylamino, or aryl, wherein the alkyl and aryl may be substituted with halo, e.g. alkyl, fluoro-substituted alkyl, dimethylamino, heteroaryl and fluoro-substituted heteroaryl such as fluoro-substituted thienyl. In some embodiments, R₇ is methyl, ethyl, i-propyl, fluoropropyl, trifluoromethyl, chlorothienyl or dimethylamino. In some embodiments, R₇ is alkyl, e.g. methyl or ethyl.

In some embodiments, R₂ is halo, OR₇ or OC(R₇)₂O. In some embodiments, R₂ is selected from the group consisting of F, Cl, OCH₃ and O(CH₂)O. In some embodiments, R₂ is OCH₃.

In some embodiments, R₃ is alkyl, including cycloalkyl-n-alkyl moieties, such as (CH₂)ₙR₅, wherein n is 3, 4, 5, 6, 7, 8, or 9 and R₅ is H or cycloalkyl. In some embodiments, R₃ is a cyclohexyl-n-alkyl moiety. In some embodiments, R₃ is cyclohexyl-n-butyl.

Some embodiments include one of the following compounds:

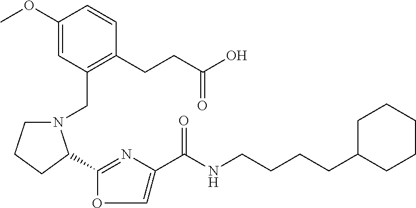

### (S)—N-(4-cyclohexylbutyl)-2-(1-(5-methoxy-2-(3-carboxypropyl)benzyl)pyrrolidin-2-yl)oxazole-4-carboxamide

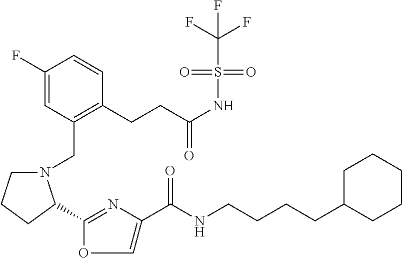

### (S)—N-(4-cyclohexylbutyl)-2-(1-(5-fluoro-2-(3-oxo-3-(trifluoromethylsulfonamido)propyl)benzyl)pyrrolidin-2-yl)oxazole-4-carboxamide

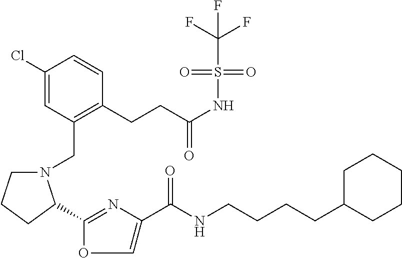

### (S)—N-(4-cyclohexylbutyl)-2-(1-(5-chloro-2-(3-oxo-3-(trifluoromethylsulfonamido)propyl)benzyl)pyrrolidin-2-yl)oxazole-4-carboxamide

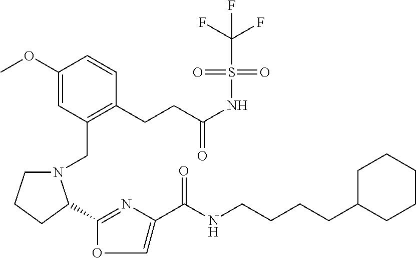

### (S)—N-(4-cyclohexylbutyl)-2-(1-(5-methoxy-2-(3-oxo-3(trifluoromethylsulfonamido)propyl)benzyl)pyrrolidin-2-yl)oxazole-4-carboxamide

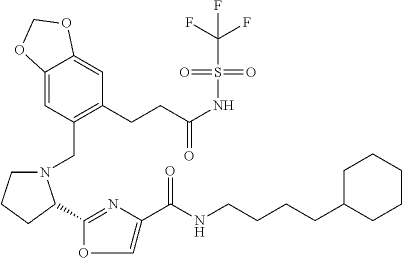

### N-(4-cyclohexylbutyl)-2-(1-{[6-(3-oxo{[(trifluoromethylsulfonamido)propyl)-1,3-benzodioxol-5-yl]methyl}pyrrolidin-2-yl)-1,3-oxazole-4-carboxamide

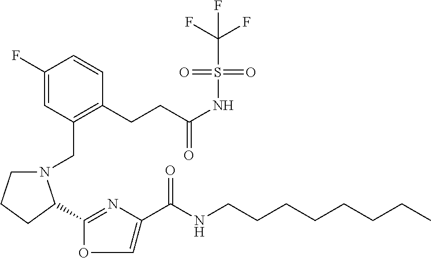

### 2-{1-[5-fluoro-2-(3-oxo-3-{[(trifluoromethyl)sulfonyl]amino}propyl)benzyl]pyrrolidin-2-yl}-N-octyl-1,3-oxazole-4-carboxamide

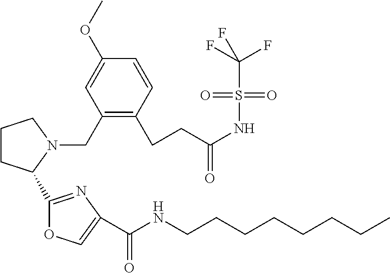

### 2-{1-[5-methoxy-2-(3-oxo-3-{[(trifluoromethyl)sulfonyl]amino}propyl)benzyl]pyrrolidin-2-yl}-N-octyl-1,3-oxazole-4-carboxamide

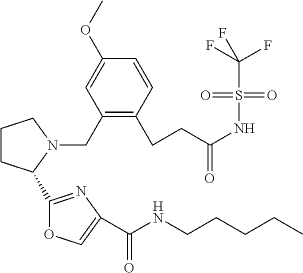

### 2-{1-(5-methoxy-2-(3-oxo-3-{[(trifluoromethyl)sulfonyl]amino]propyl)benzyl]pyrrolidin-2-yl}-N-pentyl-1,3-oxazole-4-carboxamide

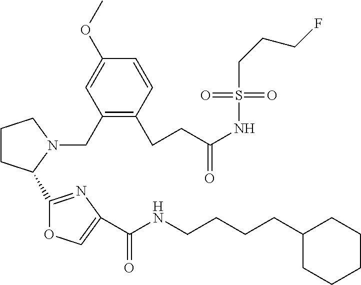

### 2(S)—N-(4-cyclohexylbutyl)-2-(1-(5-methoxy-2-(3-oxo-3-(fluoropropyl sulfonamido)propyl)benzyl)pyrrolidin-2-yl)oxazole-4-carboxamide

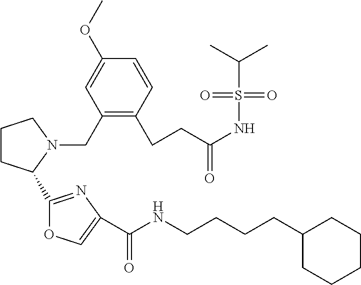

### (S)—N-(4-cyclohexylbutyl)-2-(1-(5-methoxy-2-(3-oxo-3-(isopropyl sulfonamido)propyl)benzyl)pyrrolidin-2-yl)oxazole-4-carboxamide

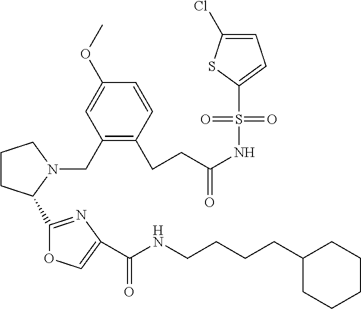

### (S)—N-(4-cyclohexylbutyl)-2-(1-(5-methoxy-2-(3-oxo-3-(5-chlorothienyl sulfonamido)propyl)benzyl)pyrrolidin-2-yl)oxazole-4-carboxamide

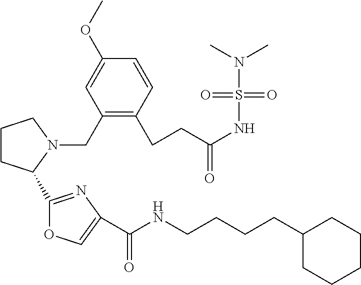

### (S)—N-(4-cyclohexylbutyl)-2-(1-(5-methoxy-2-(3-oxo-3-(N-diethyl sulfamide)propyl)benzyl)pyrrolidin-2-yl)oxazole-4-carboxamide

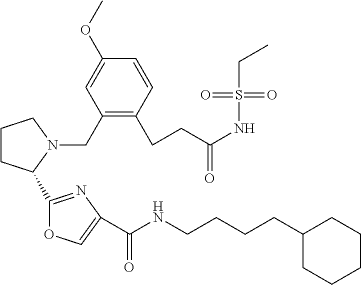

### (S)—N-(4-cyclohexylbutyl)-2-(1-(5-methoxy-2-(3-oxo-3-(ethyl sulfonamido)propyl)benzyl)pyrrolidin-2-yl)oxazole-4-carboxamide

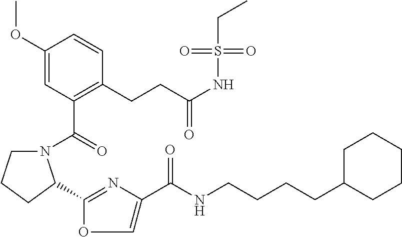

### 2-{(S)-1-[2-(3-Ethanesulfonylamino-3-oxo-propyl)-5-methoxy-benzoyl]-pyrrolidin-2-yl}-oxazole-4-carboxylic acid (4-cyclohexyl-butyl)-amide

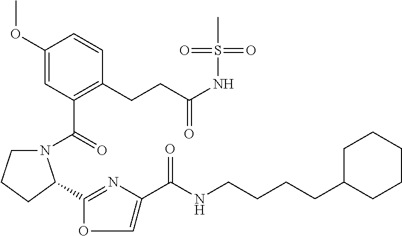

### 2-{(S)-1-[2-(3-Methanesulfonylamino-3-oxo-propyl)-5-methoxy-benzoyl]-pyrrolidin-2-yl}-oxazole-4-carboxylic acid (4-cyclohexyl-butyl)-amide

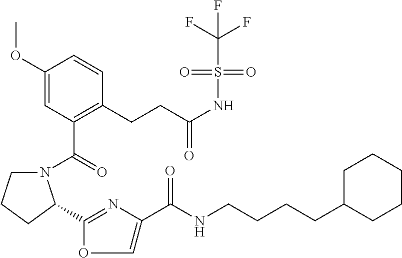

### 2-{(S)-1-[2-(3-Trifluoromethanesulfonylamino-3-oxo-propyl)-5-methoxy-benzoyl]-pyrrolidin-2-yl}-oxazole-4-carboxylic acid (4-cyclohexyl-butyl)-amide

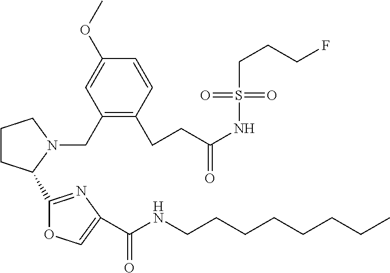

### 2-{1-(5-methoxy-2-(3-oxo-3-{[(fluoropropyl)sulfonyl]amino]propyl)benzyl]pyrrolidin-2-yl}-N-octyl-1,3-oxazole-4-carboxamide

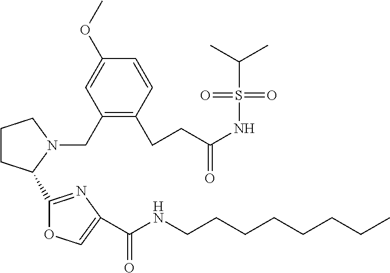

### 2-{1-(5-methoxy-2-(3-oxo-3-{[(isopropyl)sulfonyl]amino]propyl)benzyl]pyrrolidin-2-yl}-N-octyl-1,3-oxazole-4-carboxamide

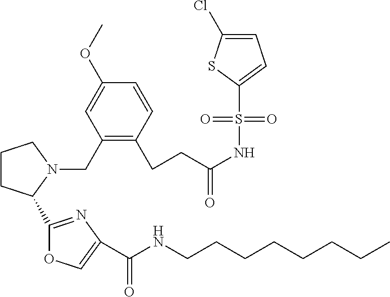

### 2-{1-(5-methoxy-2-(3-oxo-3-{[(chlorothienyl)sulfonyl)amino]propyl)benzyl]pyrrolidin-2-yl}-N-octyl-1,3-oxazole-4-carboxamide

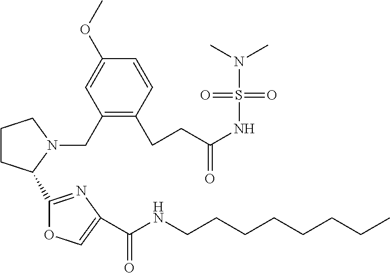

### 2-{1-(5-methoxy-2-(3-oxo-3-{[(dimethylamino)sulfonyl]amino]propyl)benzyl]pyrrolidin-2-yl}-N-octyl-1,3-oxazole-4-carboxamide

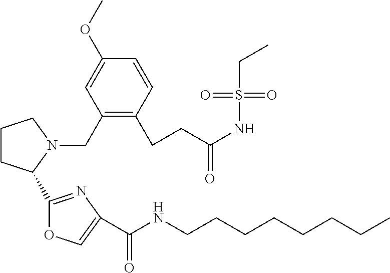

### 2-{1-(5-methoxy-2-(3-oxo-3-{[(ethyl)sulfonyl]amino]propyl)benzyl]pyrrolidin-2-yl}-N-octyl-1,3-oxazole-4-carboxamide

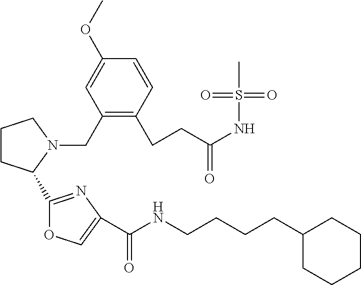

### (S)—N-(4-cyclohexylbutyl)-2-(1-(5-methoxy-2-(3-oxo-3-(methylsulfonamido)propyl)benzyl)pyrrolidin-2-yl)oxazole-4-carboxamide

Methods of treating pain, defects in cognition and locomotive activity, problems with feeding, sleeping, etc., may be carried out by treating a patient in need of the treatment with an effective amount of a compound described herein.

Some embodiments include pharmaceutical compositions containing the above compounds in combination with a pharmaceutically-acceptable excipient and to their use in medicine, in particular their use in the treatment of conditions mediated by the action of the FAAH enzyme and, additionally, ligands for the DP₁, FP, EP₁, EP₃ and EP₄ prostaglandin (PG) receptors. Some of the compounds are also useful for treating conditions mediated by the action of ligands for the thromboxane (TP) receptor.

As shown in the following tables, some of the compounds are also pan antagonists of the PG receptors, having particular activity at the FP, DP, EP₁, EP₃, EP₄ and TP receptors, but are much less active at the EP₂ and IP receptors. Thus, these compounds have a biological selectivity profile making them useful in treating diseases and conditions which are mediated by the FP, DP, EP₁, EP₃, EP₄ and TP receptors, without the potential side effects and biological limitations associated with IP and EP₂ receptor blockade.

Thus, the compounds may be also administered to treat DP₁, FP, EP₁, EP₃, TP and/or EP₄ receptor mediated diseases or conditions, as well as diseases mediated by FAAH.

For example, the condition or disease may be related to inflammation, or the DP₁, FP, EP₁, EP₃, TP and/or EP₄ receptor mediated condition or disease may be selected from: allergic conditions, asthma, allergic asthma, apnea, allergic conjunctivitis, allergic rhinitis, atopic dermatitis, uveitis, dry eye and related disorders, atherosclerosis, blood coagulation disorders, bone disorders, cancer, cellular neoplastic transformations, chronic obstructive pulmonary diseases and other forms of lung inflammation, pneumonia, congestive heart failure, diabetic retinopathy, diseases or conditions requiring a treatment of anti-coagulation, diseases requiring control of bone formation and resorption, fertility disorders, pre-term labor, endometriosis, glaucoma, hyperpyrexia, immune and autoimmune diseases, inflammatory conditions, metastic tumor growth, migraine, mucus secretion disorders, nasal congestion, nasal inflammation, occlusive vascular diseases, ocular hypertension, ocular hypotension, osteoporosis, rheumatoid arthritis, pain, perennial rhinitis, pulmonary congestion, pulmonary hypotension, Raynaud's disease, rejection in organ transplant and by-pass surgery, respiratory conditions, hirsutism, rhinorrhea, shock, sleep disorders, sleep-wake cycle disorders, and over active bladder disorders.

Compounds may be administered as a surgical adjunct in ophthalmology for cataract removal and artificial lens insertion, ocular implant procedures, photorefractive radial keratotomy and other ophthalmogical laser procedures or as a surgical adjunct in a procedure involving skin incisions, relief of pain and inflammation and scar formation/keloids post-surgery, for treating sports injuries and general aches and pains in muscles and joints. The DP₁, FP, EP₁, EP₃, TP, and/or EP₄ receptor mediated condition or disease may be an EP₁ and/or EP₄ receptor mediated condition or disease.

The DP₁, FP, EP₁, EP₃, TP and/or EP₄ receptor mediated condition or disease may be an allergic condition, e.g. an dermatological allergy, or an ocular allergy, or a respiratory allergy, e.g. nasal congestion, rhinitis, and asthma.

The condition or disease may be a bleeding disorder, or a sleep disorder, or mastocytosis.

The DP₁, FP, EP₁, EP₃, TP and/or EP₄ receptor mediated condition or disease may be associated with elevated body temperature, or ocular hypertension and glaucoma, or ocular hypotension.

In particular, the DP₁, FP, EP₁, EP₃, TP and/or EP₄ receptor mediated condition or disease may be related to pain. Therefore, the compounds may treat pain by two or more mechanisms, simultaneously, i.e. by inhibiting FAAH and antagonizing the appropriate PG receptor, simultaneously.

The pain-related condition or disease may be selected from the group consisting of arthritis, migraine, and headache.

The pain-related condition or disease may be associated with the gastrointestinal tract, wherein the condition or disease may be peptic ulcer, heartburn, reflux esophagitis, erosive esophagitis, non-ulcer dyspepsia, infection by Helicobacter pylori, alrynitis, and irritable bowel syndrome.

The pain-related condition or disease may be selected from the group consisting of hyperalgesia and allodynia, or the condition or disease may be related to mucus secretion, wherein the mucus secretion is gastrointestinal, or occurs in the nose, sinuses, throat, or lungs.

The pain-related condition or disease is related to abdominal cramping, e.g. the condition or disease may be irritable bowel syndrome.

The condition may relate to surgical procedures to treat pain, inflammation and other unwanted sequelae wherein the surgical procedure includes incision, laser surgery or implantation.

Finally, the condition may be related to pain and inflammation and post-surgical scar and keloid formation.

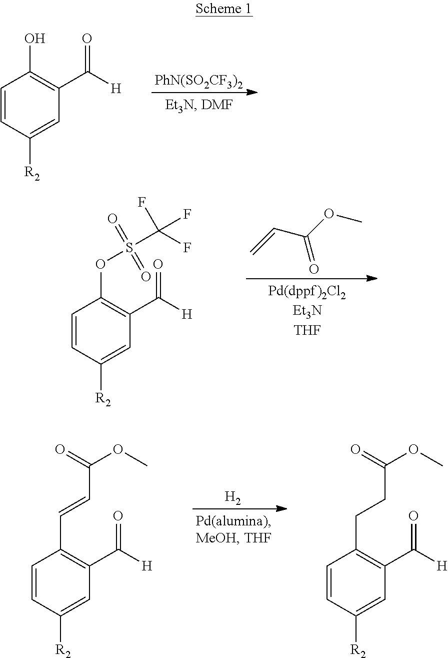

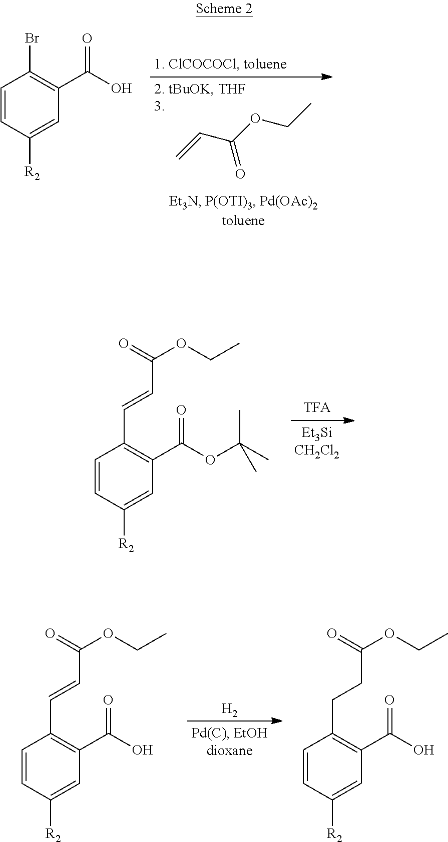

As shown in Schemes 1 and 2, certain of the compounds may be prepared by a method of making an N-alkyl-2-(1-(5-substituted-2-(3-oxo-3-(trifluoromethylsulfonamido)propyl)benzyl)pyrrolidin-2-yl)oxazole-4-carboxamide which comprises reacting the corresponding 3-(2-{2R-[4-(4-Alkylcarbamoyl)-oxazol-2-yl]-pyrrolidin-1-ylmethyl}-4-substituted-phenyl)-propionic acid with cyanuric fluoride and trifluoromethanesulfonamide to yield the N-alkyl-2-(1-(5-substituted-2-(3-oxo-3-(trifluoromethylsulfonamido)propyl)benzyl)pyrrolidin-2-yl)oxazole-4-carboxamide. In the above method, the 3-(2-{2R-[4-(4-alkylcarbamoyl)-oxazol-2-yl]-pyrrolidin-1-ylmethyl}-4-substituted-phenyl)-propionic acid may be reacted with cyanuric fluoride in the presence of pyridine, or other suitable base, at reflux, the resulting reaction mixture cooled to room temperature, diluted to separate out the organic product, preferably with ethyl acetate and water and the crude organic product is dissolved in CH₂Cl₂ and DMAP, trifluoromethanesulfonamide is added and the resulting mixture is stirred at room temperature under nitrogen or other inert gas to yield the N-alkyl-2-(1-(5-substituted-2-(3-oxo-3-(trifluoromethylsulfonamido)propyl)benzyl)pyrrolidin-2-yl)oxazole-4-carboxamide.

The 3-(2-{2R-[4-(4-alkylcarbamoyl)-oxazol-2-yl]-pyrrolidin-1-ylmethyl}-4-substituted-phenyl)-propionic acid may be made by hydrolyzing the corresponding propionic alkyl ester, i.e. 3-(2-{2R-[4-(4-Alkylcarbamoyl)-oxazol-2-yl]-pyrrolidin-1-ylmethyl}-4-substituted-phenyl)-propionic acid alkyl ester to yield the 3-(2-{2R-[4-(4-Alkylcarbamoyl)-oxazol-2-yl]-pyrrolidin-1-ylmethyl}-4-substituted-phenyl)-propionic acid.

The 3-(2-{2R-[4-(4-alkylcarbamoyl)-oxazol-2-yl]-pyrrolidin-1-ylmethyl}-4-substituted-phenyl)-propionic acid alkyl ester is made by reacting the corresponding aldehyde and proline, i.e. 2R-Pyrrolidin-2-yl-oxazole-4-carboxylic acid alkylamide may be reacted with 3-(4-substituted-2-formyl-phenyl)-propionic acid alkyl ester to yield the 3-(2-{2R-[4-(4-alkylcarbamoyl)-oxazol-2-yl]-pyrrolidin-1-ylmethyl}-4-substituted-phenyl)-propionic acid alkyl ester.

The following examples are intended to further illustrate the embodiments and include the best mode.

### Example 1

### General Method 1

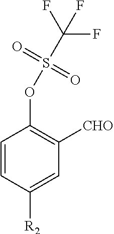

N-Phenylbis(trifluoromethanesulfonimide) (1.41 g, 3.94 mmol) was added portion-wise to a solution of the Phenol (3.57 mmol) and triethylamine (0.56 mL, 4 mmol) in DMF (3 mL) at room temperature and under nitrogen atmosphere. The resulting mixture was stirred overnight. The reaction was quenched with water (3 mL) and the mixture was extracted with diethyl ether (2×10 mL). The organic layer was dried (MgSO₄), filtered and the solvent was evaporated under vacuum.

The crude compound was purified by column in a 20 g SPE cartridge using 20% CH₂Cl₂/80% iso-hexane as eluent to give the desired triflate as a black liquid (98%).

### Example 1a

### Trifluoro-methanesulfonic acid-4-fluoro-2-formyl-phenyl ester

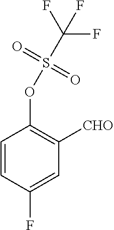

¹H-NMR (CDCl₃, 300 MHz): 10.26 (s, 1H, CHO), 7.69 (m, 1H, ArH), 7.45 (m, 2H, ArH). ¹⁹F-NMR (CDCl₃, 300 MHz) γ-73.1, −110.

### Example 1b

### Trifluoro-methanesulfonic acid-4-Chloro-2-formyl

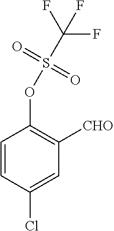

¹H-NMR (CDCl₃, 300 MHz): 10.22 (s, 1H, CHO), 7.95 (d, 1H, J=2.6 Hz, ArH), 7.68 (dd, 1H, J=2.6, 8.6 Hz, ArH), 7.38 (d, 1H, J=8.6 Hz, ArH). ¹⁹F-NMR (CDCl₃, 300 MHz) δ −73.2.

### Example 1c

### Trifluoro-methanesulfonic acid-4-methoxy-2-formyl

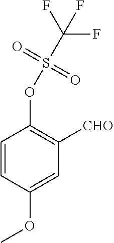

¹H-NMR (CDCl₃, 300 MHz): 10.26 (s, 1H, CHO), 7.29 (m, 3H, ArH), 3.90 (s, 3H, —OCH₃). ¹⁹F-NMR (CDCl₃, 300 MHz) δ −73.2.

### Example 2

### General Method 2

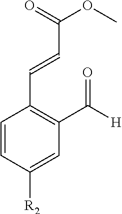

A mixture of the triflate (from General method 1) (3.37 mmol), methyl acrylate (0.70 mL), triethylamine (0.9 mL, 6.8 mmol) and Pd(dppf)₂Cl₂ (0.026 g) in THF (10 mL) was heated at reflux for 16 h under a nitrogen atmosphere. Water (10 mL) was added and the compound was extracted with ether (3×10 mL). The combined ether layers were washed with brine (10 mL), dried (MgSO4) and then evaporated to dryness under vacuum.

Then the crude compound was purified by column in a 25G Silica cartridge using 30% EtOAc/70% iso-hexane as eluent to give the conjugated ester as a light brown solid (41%).

### Example 2a

### (E)-3-(4-Fluoro-2-formyl-phenyl)-acrylic acid methyl ester

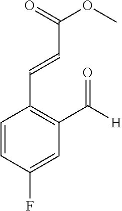

¹H-NMR (CDCl₃, 300 MHz): 10.30 (s, 1H, CHO), 8.43 (d, 1H, J=15.9 Hz, —CH═CH—CO₂CH₃), 7.61 (m, 2H, ArH), 7.34 (m, 1H, ArH), 6.37 (d, 1H, J=15.9 Hz, —CH═CH—CO₂CH₃), 3.85 (s, 3H, —CO₂CH₃). ¹⁹F-NMR (CDCl₃, 300 MHz) δ −110.

### Example 2b

### (E)-3-(4-Chloro-2-formyl-phenyl)-acrylic acid methyl ester

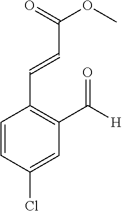

¹H-NMR (CDCl₃, 300 MHz): 10.25 (s, 1H, CHO), 8.41 (d, 1H, J=15.9 Hz, —CH═CH—CO₂CH₃), 7.84 (s, 1H, ArH), 7.88 (s, 2H, ArH), 6.37 (d, 1H, J=15.9 Hz, —CH═CH—CO₂CH₃), 3.82 (s, 3H, —CO₂CH₃).

### Example 2c

### (E)-3-(4-Methoxy-2-formyl-phenyl)-acrylic acid methyl ester

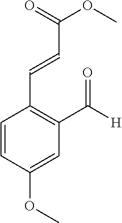

¹H-NMR (CDCl₃, 300 MHz): 10.35 (s, 1H, CHO), 8.47 (d, 1H, J=15.9 Hz, —CH═CH—CO₂CH₃), 7.61 (d, 1H, J=8.6 Hz, ArH), 7.39 (s, 1H, ArH), 7.16 (m, 1H, ArH), 6.33 (d, 1H, J=15.9 Hz, —CH═CH—CO₂CH₃), 3.91 (s, 3H, —OCH₃), 3.83 (s, 3H, —CO₂CH₃).

### Example 2d

### (E)-3-(6-Formyl-benzo[1,3]dioxol-5-yl)-acrylic acid methyl ester

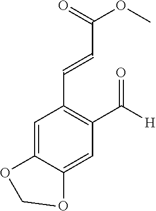

This derivative was prepared following General Method 2 but starting from the commercially available aromatic bromide.

¹H-NMR (CDCl₃, 300 MHz): 10.27 (s, 1H, CHO), 8.45 (d, 1H, J=15.9 Hz, —CH═CH—CO₂CH₃), 7.37 (s, 1H, ArH), 7.07 (s, 1H, ArH), 6.33 (d, 1H, J=15.9 Hz, —CH═CH—CO₂CH₃), 612 (s, 2H, —OCH₂O—), 3.85 (s, 3H, —CO₂CH₃).

### Example 3

### General Method 3

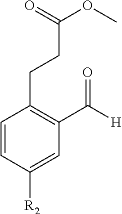

The unsaturated methyl ester (from General method 2) (0.3 mmol) was dissolved in a mixture of THF (2 mL) and MeOH (4 mL). Palladium on Alumina catalyst (35 mg) was added and the suspension was stirred for 1.5 h at room temperature under a hydrogen atmosphere.

The catalyst was removed by filtration through Hyflo and the filtrate was evaporated under vacuum to give a yellow solid (70%).

### Example 3a

### 3-(4-Fluoro-2-formyl-phenyl)-propionic acid methyl ester

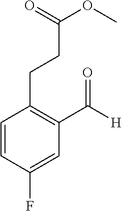

¹H-NMR (CDCl₃, 300 MHz): 10.30 (s, 1H, CHO), 7.61 (m, 2H, ArH), 7.34 (m, 1H, ArH), 3.85 (s, 3H, —CO₂CH₃), 2.88 (m, 2H, ArCH₂CH₂CO₂Me), 2.63 (m, 2H, ArCH₂CH₂CO₂Me). ¹⁹F-NMR (CDCl₃, 300 MHz) δ −110

### Example 3b

### 3-(4-Chloro-2-formyl-phenyl)-propionic acid methyl ester

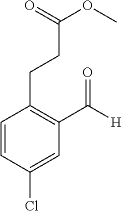

¹H-NMR (CDCl₃, 300 MHz): 10.25 (s, 1H, CHO), 7.84 (s, 1H, ArH), 7.88 (s, 2H, ArH), 3.82 (s, 3H, —CO₂CH₃), 2.87 (m, 2H, ArCH₂CH₂CO₂Me), 2.59 (m, 2H, ArCH₂CH₂CO₂Me).

### Example 3c

### 3-(4-Methoxy-2-formyl-phenyl)-propionic acid methyl ester

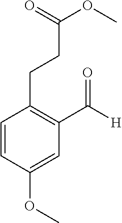

¹H-NMR (CDCl₃, 300 MHz): 10.35 (s, 1H, CHO), 7.61 (d, 1H, J=8.6 Hz, ArH), 7.39 (s, 1H, ArH), 7.16 (m, 1H, ArH), 3.91 (s, 3H, —OCH₃), 3.83 (s, 3H, —CO₂CH₃), 2.92 (m, 2H, ArCH₂CH₂CO₂Me), 2.61 (m, 2H, ArCH₂CH₂CO₂Me).

### Example 3d

### 3-(6-Formyl-benzo[1,3]dioxol-5-yl)-propionic acid methyl ester

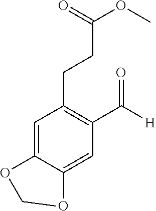

¹H-NMR (CDCl₃, 300 MHz): 10.27 (s, 1H, CHO), 7.37 (s, 1H, ArH), 7.07 (s, 1H, ArH), 612 (s, 2H, —OCH₂O—), 3.85 (s, 3H, —CO₂CH₃), 2.93 (m, 2H, ArCH₂CH₂CO₂Me), 2.63 (m, 2H, ArCH₂CH₂CO₂Me).

### Example 4

### General Method 4

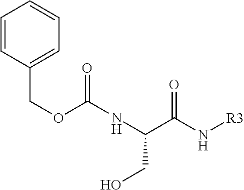

A solution of Z-protected-L-serine (5 g, 20.9 mmol), amine (25.1 mmol), WSC (6 g, 31.4 mmol), N-methylmorpholine (2.55 mL, 23 mmol) in DMF (150 mL) was stirred at room temperature for 16 h under a nitrogen atmosphere.

The reaction mixture was evaporated to dryness under vacuum and the residue was re-dissolved in EtOAc (100 mL). This solution was washed with 2M solution of HCl (2×75 mL), sat. Solution of sodium bicarbonate (2×75 mL), brine (2×75 mL) and dried (Na₂SO₄). The solvent was evaporated to give the Z-protected serine amide as a white solid (64%).

### Example 4a

### (2-Hydroxy-1-octylcarbamoyl-ethyl)-carbamic-acid benzyl ester

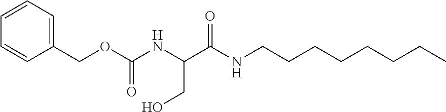

¹H-NMR (CDCl₃, 300 MHz): 7.37 (m, 5H, ArH), 6.56 (m, 1H, NH), 5.83 (m, 1H, NH), 5.15 (s, 2H, ArCH₂—), 4.16 (m, 2H, CH₂OH), 3.67 (m, 1H, NHCHCO), 3.24 (m, 2H, CONHCH₂—), 1.49 (m, 2H, NHCH₂—CH₂—), 1.27 (m, 10H, —CH₂—CH₂—), 0.89 (m, 3H, —CH₃)

### Example 5

### General Method 5

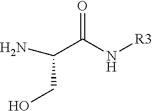

The Z-protected serinamide (from General method 4) (0.98 mmol) was dissolved in a mixture of THF (25 mL) and MeOH (18 mL). Then Pd(OH)₂ (52 mg) was added and the reaction mixture was stirred for 16 h at room temperature under a hydrogen atmosphere.

The palladium hydroxide was removed by filtration through Hyflo and the filtrate was evaporated under vacuum to give the free serine amide as a yellow solid (98%).

### Example 5a

### 2-Amino-3-hydroxy-N-octyl-propionamide

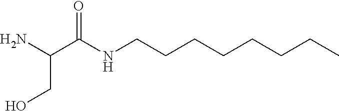

¹H-NMR (CDCl₃, 300 MHz): 3.84-3.73 (m, 2H, CH₂OH), 3.47 (m, 1H, NHCHCO), 3.26 (m, 2H, CONHCH₂—), 2.49 (bs, 2H, NH₂), 1.52 (m, 2H, NHCH₂—CH₂—), 1.29 (m, 10H, —CH₂—CH₂—), 0.89 (m, 3H, —CH₃).

### Example 6

### General Method 6

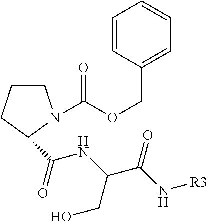

To a solution N-benzyloxicarbonyl-L-proline (14.86 mmol) and free serine amide (from General Method 5) (16.35 mmol) in dimethylformamide (150 mL) under a nitrogen atmosphere, N-methylmorpholine (3.6 mL, 32.7 mmol) was added, followed by HBTU (6.2 g, 16.35 mmol). The resulting mixture was stirred at room temperature for 16 h.

After this time the solution was concentrated under vacuum and the residue was dissolved in ethyl acetate (100 mL). The solution was washed with 2M HCl solution (100 mL), a saturated solution of NaHCO₃ (100 mL) and dried over MgSO₄. Filtration and concentrated under vacuum yield the desired compound as a thick oil.

### Example 6a

### 2R-(2-Hydroxy-1-octylcarbamoyl-ethylcarbamoyl)-pyrrolidine-1-carboxylic acid benzyl ester

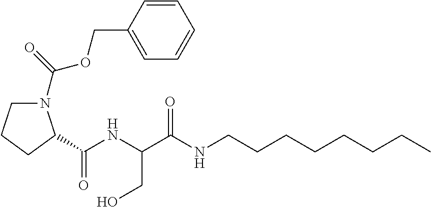

¹H-NMR (CDCl₃, 300 MHz): 7.35 (m, 5H, ArH), 5.15 (s, 2H, ArCH₂—), 4.48 (m, 1H, NCHCONH), 4.33 (m, 2H, CH₂OH), 4.07 (m, 1H, NHCHCO), 3.59 (m, 2H, CH₂NCO), 3.19 (m, 2H, CONHCH₂—), 2.20 (m, 2H, —CH₂—CH₂—), 1.94 (m, 2H, —CH₂—CH₂—), 1.49 (m, 2H, NHCH₂—CH₂—), 1.27 (m, 10H, —CH₂—CH₂—), 0.88 (m, 3H, —CH₃).

### Example 7

### General Method 7

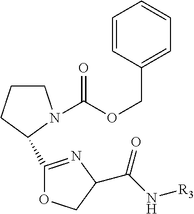

To a solution of amide (from General Method 6) (14.86 mmol) in dichloromethane (200 mL), at −25° C. under a nitrogen atmosphere, 40% solution of deoxo-fluor (17.09 mmol) was added and the resulting mixture was stirred at room temperature for 2.5 h.

After this time, a saturated solution of NaHCO₃ (200 mL) was added and the mixture was diluted with more CH₂Cl₂ (100 mL). The organic layer was separated, then washed with saturated brine (150 mL), and dried over MgSO₄. Filtration and concentrated in vacuo yielded the crude compound as a thick oil.

The residue was purified by column chromatography on silica using a solvent gradient starting from ethyl acetate/iso-hexane 1:1 to ethyl acetate/methanol 9:1, to isolate the title compound as a thick oil (72%).

### Example 7a

### 2R-(4-Octylcarbamoyl-4,5-dihydro-oxazol-2-yl)-pyrrolidine-1-carboxylic acid benzyl ester

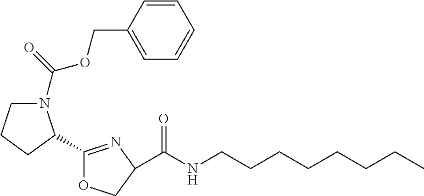

¹H-NMR (CDCl₃, 300 MHz): 7.37 (m, 5H, ArH), 5.12 (s, 2H, ArCH₂—), 4.70-4.30 (m, 4H, NCHCONH+CH₂O—+NHCHCO), 3.55 (m, 2H, CH₂NCO), 3.22 (m, 2H, CONHCH₂—), 2.22 (m, 1H, —CH₂—CH₂—), 2.05 (m, 3H, —CH₂—CH₂—), 1.53 (m, 2H, NHCH₂—CH₂—), 1.26 (m, 10H, —CH₂—CH₂—), 0.88 (m, 3H, —CH₃).

### Example 8

### General Method 8

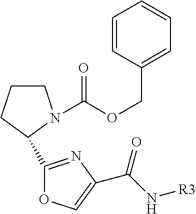

To a suspension of copper bromide (7.48 mmol) in degassed dichloromethane (21 mL), under nitrogen atmosphere and in a water bath, was added HMTA (7.48 mmol) followed by DBU (7.48 mmol) and the resulting mixture was stirred for 15 minutes. Then, a solution of oxazolidine (from general method 7) (1.87 mmol) in dichloromethane (11 mL) was added and the resulting mixture was stirred at room temperature for 16 h.

After this time, the solution was concentrated under vacuum and the residue was partitioned between ethyl acetate (30 mL) and a 1:1 sat. solution of NH₄Cl and NH₃ (30 mL). Then, the organic layer was separated and washed with Brine (30 mL), and dried over MgSO₄. Filtration and concentrated in vacuo yield the crude compound as a thick oil.

The residue was purified by column chromatography on a 10 g silica SPE using ethyl acetate/iso-hexane 40%:60% to isolate the title compound as a yellow solid (80%).

### Example 8a

### 2R-(4-Octylcarbamoyl-oxazol-2-yl)-pyrrolidine-1-carboxylic acid benzyl ester

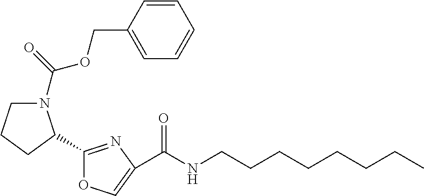

¹H-NMR (CDCl₃, 300 MHz): 8.10 (s, 1H, ═CH), 8.01 (s, 1H, ═CH), 7.37 (m, 7H, ArH), 7.13 (m, 3H, ArH), 6.88-6.79 (m, 2H, NH), 5.21-4.95 (m, 8H, NCHCONH+PhCH₂O—+NHCHCO), 3.70 (m, 4H, CH₂NCO), 3.59 (m, 4H, CONHCH₂—), 2.30 (m, 2H, —CH₂—CH₂—), 2.06 (m, 6H, —CH₂—CH₂—), 1.61 (m, 4H, NHCH₂—CH₂—), 1.29 (m, 20H, —CH₂—CH₂—), 0.88 (m, 6H, —CH₃)

### Example 9

### General Method 9

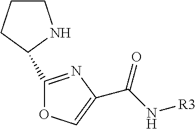

The Z-protected oxazole (from General Method 8) (0.98 mmol) was dissolved in MeOH (25 mL) then Pd(OH)₂ (52 mg) was added and the suspension was stirred overnight at room temperature under hydrogen. The palladium hydroxide was removed by filtration through Hyflo and the filtrate was evaporated under vacuum to give a yellow solid (95%).

### Example 9a

### 2R-Pyrrolidin-2-yl-oxazole-4-carboxylic acid octylamide

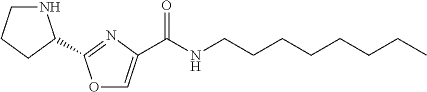

¹H-NMR (CDCl₃, 300 MHz): 8.15 (s, 1H, ═CH), 7.03 (m, 1H, NH), 4.46 (m, 1H, NCH-Oxazole), 3.39 (dd, 2H, J=7, 14 Hz, CONHCH₂—), 3.24 (m, 2H, —CH₂N—), 2.30-1.88 (m, 4H, —CH₂—CH₂—), 1.59 (m, 2H, NHCH₂—CH₂—), 1.28 (m, 10H, —CH₂—CH₂—), 0.88 (m, 3H, —CH₃).

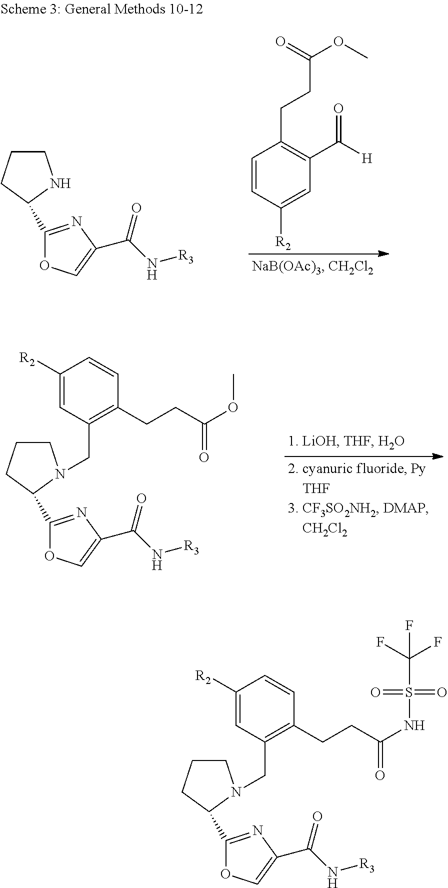

### General Method 10

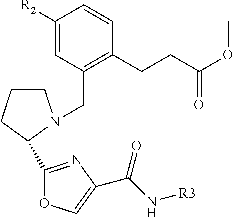

To a solution of aldehyde (from General Method 3) (1.49 mmol) and free proline (from general method 9) (1.24 mmol) in CH₂Cl₂ (15 mL) was added sodium triacetoxyborohydride (0.369 g, 1.74 mmol). The mixture was stirred under a nitrogen atmosphere for 16 hours at room temperature.

The mixture was diluted with 15 mL of CH₂Cl₂ and water was added. The organic layer was separated, washed with saturated brine (30 mL), dried (Na₂SO₄) and the solvent was evaporated to give required product as a yellow solid (85%).

### Example 10a

### 3-(2-{2R-[4-(4-Cyclohexyl-butylcarbamoyl)-oxazol-2-yl]-pyrrolidin-1-ylmethyl}-4-fluoro-phenyl)-propionic acid methyl ester

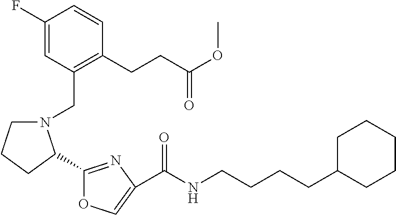

¹H-NMR (CDCl₃, 300 MHz): 8.13 (s, 1H, ═CH), 7.55 (dd, 1H, J=5.5, 8.4 Hz, ArH), 7.17 (m, 1H, NH), 7.05 (dd, 1H, J=2.6, 9.5 Hz, ArH), 6.96 (dt, 1H, J=2.6, 8.4, ArH), 4.03 (d, 1H, J=11.9 Hz, —NCH₂Ar), 3.81 (s, 3H, —CO₂CH₃), 3.76 (t, 1H, J=7.7 Hz, NCH-Oxazole), 3.53 (d, 1H, J=11.9 Hz, —NCH₂Ar), 3.41 (dd, 2H, J=7, 14 Hz, CONHCH₂—), 3.10 (m, 2H, ArCH₂CH₂CO₂Me), 3.00 (m, 1H, —CH₂N—), 2.70 (m, 2H, ArCH₂CH₂CO₂Me), 2.40 (m, 1H, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—). ¹⁹F-NMR (CDCl₃, 300 MHz) δ −111.

### Example 10b

### 3-(2-{2R-[4-(4-Cyclohexyl-butylcarbamoyl)-oxazol-2-yl]-pyrrolidin-1-ylmethyl}-4-chloro-phenyl)-propionic acid methyl ester

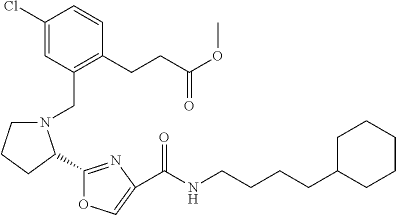

¹H-NMR (CDCl₃, 300 MHz): 8.18 (d, 1H, J=15.9 Hz, —CH═CH—CO₂Me), 8.12 (s, 1H, ═CH), 7.47 (m, 1H, ArH), 7.29 (m, 1H, ArH), 7.22 (m, 2H, ArH+NH), 6.31 (d, 1H, J=15.9 Hz, —CH═CH—CO₂Me), 3.97 (d, 1H, J=11.9 Hz, —NCH₂Ar), 3.80 (s, 3H, —CO₂CH₃), 3.73 (t, 1H, J=7.7 Hz, NCH-Oxazole), 3.52 (d, 1H, J=11.9 Hz, —NCH₂Ar), 3.40 (dd, 2H, J=7, 14 Hz, CONHCH₂—), 3.05 (m, 2H, ArCH₂CH₂CO₂Me), 2.99 (m, 1H, —CH₂N—), 2.71 (m, 2H, ArCH₂CH₂CO₂Me), 2.41 (m, 1H, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—).

### Example 10e

### 3-(2-{2R-[4-(4-Cyclohexyl-butylcarbamoyl)-oxazol-2-yl]-pyrrolidin-1-ylmethyl}-4-methoxy-phenyl)-propionic acid methyl ester

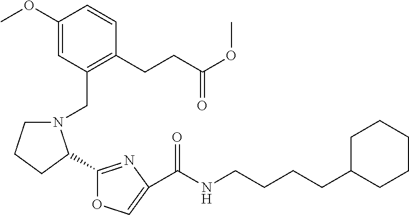

¹H-NMR (CDCl₃, 300 MHz): 8.23 (d, 1H, J=15.9 Hz, —CH═CH—CO₂Me), 8.14 (s, 1H, ═CH), 7.55 (d, 1H, J=8.4 Hz, ArH), 7.24 (m, 1H, NH), 6.82 (m, 2H, ArH), 6.27 (d, 1H, J=15.9 Hz, —CH═CH—CO₂Me), 4.02 (d, 1H, J=11.9 Hz, —NCH₂Ar), 3.82 (s, 3H, Ar—OCH₃), 3.80 (s, 3H, —CO₂CH₃), 3.72 (t, 1H, J=7.7 Hz, NCH-Oxazole), 3.50 (d, 1H, J=11.9 Hz, —NCH₂Ar), 3.41 (dd, 2H, J=7, 14 Hz, CONHCH₂—), 3.15 (m, 2H, ArCH₂CH₂CO₂Me), 2.99 (m, 1H, —CH₂N—), 2.70 (m, 2H, ArCH₂CH₂CO₂Me), 2.41 (c, 1H, J=8.6 Hz, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—).

### Example 10d

### 3-(6-{2R-[4-(4-Cyclohexyl-butylcarbamoyl)-oxazol-2-yl]-pyrrolidin-1-ylmethyl}-benzo[1,3]dioxol-5-yl)-propionic acid methyl ester

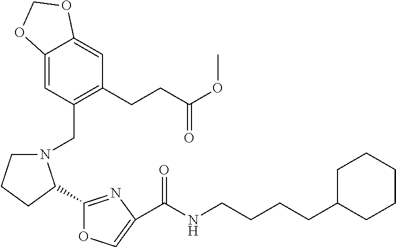

¹H-NMR (CDCl₃, 300 MHz): 8.18 (d, 1H, J=15.9 Hz, —CH═CH—CO₂Me), 8.16 (s, 1H, ═CH), 7.28 (m, 1H, NH), 7.04 (s, 1H, ArH), 6.76 (s, 1H, ArH), 6.21 (d, 1H, J=15.9 Hz, —CH═CH—CO₂Me), 5.96 (s, 2H, —OCH₂O—), 3.96 (d, 1H, J=11.9 Hz, —NCH₂Ar), 3.72 (t, 1H, J=7.7 Hz, NCH-Oxazole), 3.43 (d, 1H, J=11.9 Hz, —NCH₂Ar), 3.40 (dd, 2H, J=7, 14 Hz, CONHCH₂—), 2.96 (m, 1H, —CH₂N—), 2.85 (m, 2H, ArCH₂CH₂CO₂Me), 2.69 (m, 2H, ArCH₂CH₂CO₂Me), 2.37 (c, 1H, J=8.6 Hz, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—).

### Example 10e

### 3-(2-{2R-[4-(octylcarbamoyl)-oxazol-2-yl]-pyrrolidin-1-ylmethyl}-4-fluoro-phenyl)-propionic acid methyl ester

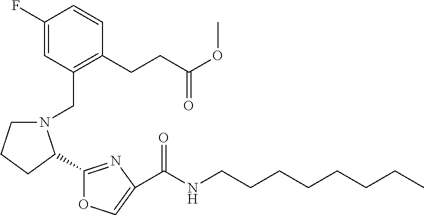

¹H-NMR (CDCl₃, 300 MHz): 8.09 (s, 1H, ═CH), 7.08 (dd, 1H, J=5.5, 8.4 Hz, ArH), 7.01 (m, 1H, NH), 6.98 (dd, 1H, J=2.6, 9.5 Hz, ArH), 6.97 (dt, 1H, J=2.6, 8.4, ArH), 3.88 (d, 1H, J=11.9 Hz, —NCH₂Ar), 3.76 (t, 1H, J=7.7 Hz, NCH-Oxazole), 3.68 (s, 3H, —CO₂CH₃), 3.42 (d, 1H, J=11.9 Hz, —NCH₂Ar), 3.41 (dd, 2H, J=7, 14 Hz, CONHCH₂—), 3.10 (m, 2H, ArCH₂CH₂CO₂Me), 3.00 (m, 2H, —CH₂N—), 2.70 (m, 2H, ArCH₂CH₂CO₂Me), 2.69-1.85 (m, 4H, —CH₂—CH₂—), 1.71-1.55 (m, 2H, NHCH₂—CH₂—), 1.36 (m, 2H, —CH₂—CH₂—), 1.25-1.19 (m, 8H, —CH₂—CH₂—), 0.89 (m, 3H, —CH₃). ¹⁹F-NMR (CDCl₃, 300 MHz) δ −111.

### Example 11

### General method 11

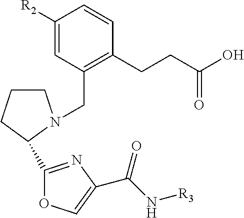

The ester (from General Method 10) (1.82 mmol) was dissolved in THF (20 mL) and a solution of LiOH (0.302 g, 7.3 mmol) in water (10 mL) was added. The resulting mixture was heated at 60° C. for 16 h.

Then, EtOAc was added (10 mL) and the solution was neutralized with a 2M solution of HCl. The organic layer was separated, washed with brine (10 mL) and dried (Na₂SO₄). The mixture was filtered and the solvent was evaporated to give crude product.

The compound was purified by column chromatography on a 10 g SPE cartridge, using as eluent: 2% MeOH/98% CH₂Cl₂, to give the carboxylic acid as a white solid (70%).

### Example 11a

### 3-(2-{2R-[4-(4-Cyclohexyl-butylcarbamoyl)-oxazol-2-yl]-pyrrolidin-1-ylmethyl}-4-fluoro-phenyl)-propionic acid

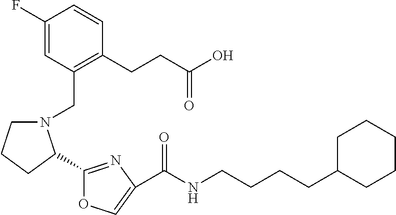

¹H-NMR (CDCl₃, 300 MHz): 8.18 (s, 1H, ═CH), 7.10 (m, 2H, ArH+NH), 6.97 (m, 1H, ArH), 6.88 (m, 1H, ArH), 3.90 (d, 1H, J=11.9 Hz, —NCH₂Ar), 3.77 (t, 1H, J=7.7 Hz, NCH-Oxazole), 3.40 (m, 3H, —NCH₂Ar+CONHCH₂—), 2.99 (m, 1H, —CH₂N—), 2.88 (m, 2H, ArCH₂CH₂CO₂H), 2.59 (m, 2H, ArCH₂CH₂CO₂H), 2.41 (m, 1H, —CH₂N—), 2.24-1.90 (m, 4H, —CH₂—CH₂—), 1.60 (m, 2H, NHCH₂—CH₂—), 1.27 (m, 10H, —CH₂—CH₂—), 0.88 (m, 5H, —CH₂—CH₂—).

¹⁹F-NMR (CDCl₃, 300 MHz) δ −111

### Example 11b

### 3-(2-{2R-[4-(4-Cyclohexyl-butylcarbamoyl)-oxazol-2-yl]-pyrrolidin-1-ylmethyl}-4-chloro-phenyl)-propionic acid

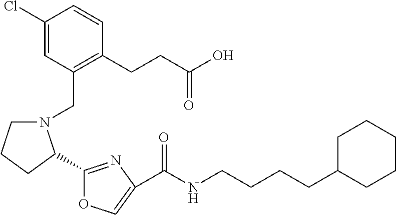

¹H-NMR (CDCl₃, 300 MHz): 8.16 (s, 1H, ═CH), 7.19 (m, 2H, ArH), 7.09 (d, 1H, J=8.4 Hz, ArH), 7.02 (m, 1H, NH), 3.91 (d, 1H, J=11.9 Hz, —NCH₂Ar), 3.76 (t, 1H, J=7.7 Hz, NCH-Oxazole), 3.40 (m, 3H, —NCH₂Ar+CONHCH₂—), 3.00 (m, 1H, —CH₂N—), 2.87 (m, 2H, ArCH₂CH₂CO₂H), 2.60 (m, 2H, ArCH₂CH₂CO₂H), 2.41 (m, 1H, —CH₂N—), 2.24-1.90 (m, 4H, —CH₂—CH₂—), 1.60 (m, 2H, NHCH₂—CH₂—), 1.27 (m, 10H, —CH₂—CH₂—), 0.88 (m, 5H, —CH₂—CH₂—).

### Example 11c

### 3-(2-{2R-[4-(4-Cyclohexyl-butylcarbamoyl)-oxazol-2-yl]-pyrrolidin-1-ylmethyl}-4-methoxy-phenyl)-propionic acid

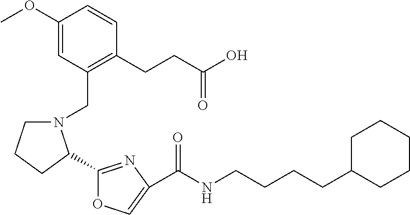

¹H-NMR (CDCl₃, 300 MHz): 8.18 (s, 1H, ═CH), 7.07 (m, 2H, ArH+NH), 6.77 (m, 2H, ArH), 3.91 (d, 1H, J=11.9 Hz, —NCH₂Ar), 3.77 (s, 3H, ArOCH₃), 3.77 (t, 1H, J=7.7 Hz, NCH-Oxazole), 3.38 (m, 3H, —NCH₂Ar+CONHCH₂—), 3.00 (m, 1H, —CH₂N—), 2.85 (m, 2H, ArCH₂CH₂CO₂H), 2.59 (m, 2H, ArCH₂CH₂CO₂H), 2.41 (m, 1H, —CH₂N—), 2.24-1.90 (m, 4H, —CH₂—CH₂—), 1.60 (m, 2H, NHCH₂—CH₂—), 1.27 (m, 10H, —CH₂—CH₂—), 0.88 (m, 5H, —CH₂—CH₂—).

### Example 11c

### 3-(6-{2R-[4-(4-Cyclohexyl-butylcarbamoyl)-oxazol-2-yl]-pyrrolidin-1-ylmethyl}-benzo[1,3]dioxol-5-yl)-propionic acid

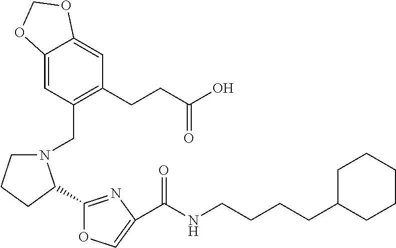

¹H-NMR (CDCl₃, 300 MHz): 8.19 (s, 1H, ═CH), 7.11 (m, 1H, NH), 6.69 (s, 1H, ArH), 6.64 (s, 1H, ArH), 5.89 (s, 2H, —OCH₂O—), 3.84 (d, 1H, J=11.9 Hz, —NCH₂Ar), 3.72 (t, 1H, J=7.7 Hz, NCH-Oxazole), 3.39 (dd, 2H, J=7, 14 Hz, CONHCH₂—), 3.29 (d, 1H, J=11.9 Hz, —NCH₂Ar), 3.00 (m, 1H, —CH₂N—), 2.79 (m, 2H, ArCH₂CH₂CO₂H), 2.60 (m, 2H, ArCH₂CH₂CO₂H), 2.38 (c, 1H, J=8.6 Hz, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—).

### Example 11d

### 3-(2-{2R-[4-(octylcarbamoyl)-oxazol-2-yl]-pyrrolidin-1-ylmethyl}-4-fluoro-phenyl)-propionic acid

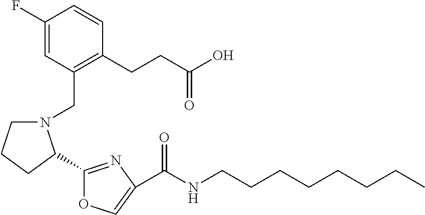

¹H-NMR (CDCl₃, 300 MHz): 8.16 (s, 1H, ═CH), 7.09 (m, 2H, ArH+NH), 6.95 (dd, 1H, J=2.6, 9.5 Hz, ArH), 6.82 (dt, 1H, J=2.6, 8.4, ArH), 3.85 (d, 1H, J=11.9 Hz, —NCH₂Ar), 3.73 (t, 1H, J=7.7 Hz, NCH-Oxazole), 3.36 (m, 3H, —NCH₂Ar+CONHCH₂—), 2.86 (m, 3H, ArCH₂CH₂CO₂Me+—CH₂N—), 2.60 (m, 2H, ArCH₂CH₂CO₂Me), 2.36 (m, 1H, —CH₂N), 2.24-1.80 (m, 4H, —CH₂—CH₂—), 1.71-1.55 (m, 4H, NHCH₂—CH₂—), 1.25-1.19 (m, 8H, —CH₂—CH₂—), 0.89 (m, 3H, —CH₃).

¹⁹F-NMR (CDCl₃, 300 MHz) δ −111.

### Example 12

### General Method 12

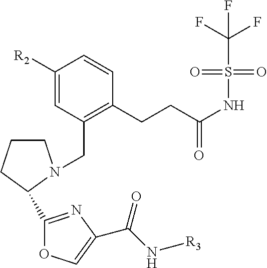

To a solution of saturated acid (from General Method 11) (0.15 mmol) in THF (7 mL), under nitrogen atmosphere, was added pyridine (0.45 mmol) and cyanuric fluoride (1.125 mmol) and the resulting mixture was refluxed for 4 h. The reaction mixture was left to cool to room temperature, then diluted with ethyl acetate (15 mL) and water (10 mL). The organic layer was separated, washed with a saturated solution of NaHCO₃ (10 mL), then saturated brine (10 mL), dried (MgSO₄), filtered and the solvent was evaporated under vacuum.

The crude product was re-dissolved in CH₂Cl₂ (7 mL) and DMAP (0.6 mmol) and trifluoromethanesulfonamide (0.45 mmol) were added. The resulting mixture was stirred at room temperature under nitrogen for 16 h.

After this time, the reaction mixture was diluted with more CH₂Cl₂ (15 mL) and water (10 mL) was added. The organic layer was separated, washed with a 2M solution of HCl (5 mL), then saturated brine (10 mL) and dried (MgSO₄), filtered and the solvent was evaporated under vacuum.

The residue was purified by column chromatography through a 10 g SPE silica cartridge using a solvent gradient starting from ethyl acetate to ethyl acetate/methanol 9:1, to isolate the title compound as thick oil (60%).

### Example 12a

### S)—N-(4-cyclohexylbutyl)-2-(1-(5-fluoro-2-(3-oxo-3-(trifluoromethylsulfonamido)propyl)benzyl)pyrrolidin-2-yl)oxazole-4-carboxamide

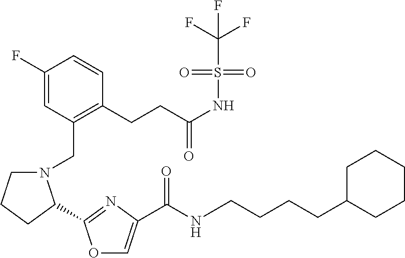

¹H-NMR (CDCl₃, 300 MHz): 8.27 (s, 1H, ═CH), 7.24 (m, 1H, NH), 7.05 (dd, 1H, J=6, 8.4 Hz, ArH), 6.85 (m, 2H, ArH), 3.91 (d, 1H, J=11.9 Hz, —NCH₂Ar), 3.74 (t, 1H, J=7.7 Hz, NCH-Oxazole), 3.36 (m, 2H, CONHCH₂—), 3.23 (d, 1H, —NCH₂Ar), 2.99 (m, 1H, —CH₂N—), 2.59 (m, 4H, ArCH₂CH₂CO₂H), 2.49 (m, 1H, —CH₂N—), 2.40-2.20 (m, 4H, —CH₂—CH₂—), 1.90 (m, 2H, NHCH₂—CH₂—), 1.27 (m, 10H, —CH₂—CH₂—), 0.88 (m, 5H, —CH₂—CH₂—).

¹⁹F-NMR (CDCl₃, 300 MHz) δ −79, −118

LC-MS (M⁺+1) 631.

### Example 12b

### (S)—N-(4-cyclohexylbutyl)-2-(1-(5-chloro-2-(3-oxo-3-(trifluoromethylsulfonamido)propyl)benzyl)pyrrolidin-2-yl)oxazole-4-carboxamide

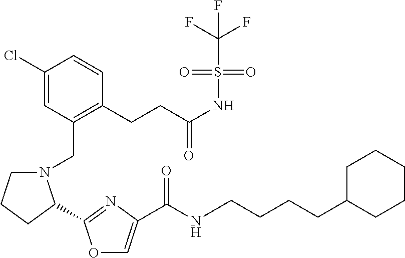

¹H-NMR (CDCl₃, 300 MHz): 8.33 (s, 1H, ═CH), 7.15 (m, 2H, ArH), 7.04 (m, 1H, ArH), 7.02 (m, 1H, NH), 4.01 (m, 2H, —NCH₂Ar+NCH-Oxazole), 3.39 (m, 3H, CONHCH₂—+—NCH₂Ar), 3.15 (m, 1H, —CH₂N—), 2.70-2.46 (m, 5H, ArCH₂CH₂CONH+—CH₂N—), 2.29 (m, 2H, —CH₂—CH₂—), 2.03 (m, 2H, —CH₂—CH₂—), 1.90 (m, 2H, NHCH₂—CH₂—), 1.27 (m, 10H, —CH₂—CH₂—), 0.88 (m, 5H, —CH₂—CH₂—). ¹⁹F-NMR (CDCl₃ −79δ, 300 MHz). LC-MS (M⁺+1) 647.

### Example 12c

### (S)—N-(4-cyclohexylbutyl)-2-(1-(5-methoxy-2-(3-oxo-3-(trifluoromethylsulfonamido)propyl)benzyl)pyrrolidin-2-yl)oxazole-4-carboxamide

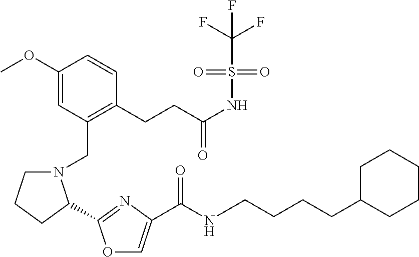

¹H-NMR (CDCl₃, 300 MHz): 8.23 (s, 1H, ═CH), 7.06 (m, 2H, ArH), 6.75 (m, 2H, ArH+NH), 3.95 (d, 1H, J=11.9 Hz, —NCH₂Ar), 3.77 (m, 4H, NCHOxazole+ArOCH₃), 3.41 (m, 2H, CONHCH₂), 3.25 (d, 1H, J=11.9 Hz, —NCH₂Ar), 3.01 (m, 1H, —CH₂N—), 2.71 (m, 3H, ArCH₂CH₂CO₂H), 2.71 (m, 3H, ArCH₂CH₂CO₂H), 2.51 (m, 1H, ArCH₂CH₂CO₂H), 2.41 (m, 1H, —CH₂N—), 2.21 (m, 2H, —CH₂—CH₂—), 1.97 (m, 2H, —CH₂—CH₂—), 1.90 (m, 2H, NHCH₂—CH₂—), 1.27 (m, 10H, —CH₂—CH₂—), 0.88 (m, 5H, —CH₂—CH₂—). ¹⁹F-NMR (CDCl₃, 300 MHz) δ −79. LC-MS (M⁺+1) 643.

### Example 12d

### N-(4-cyclohexylbutyl)-2-(1-{[6-(3-oxo{[(trifluoromethylsulfonamido)propyl)-1,3-benzodioxol-5-yl]methyl}pyrrolidin-2-yl)-1,3-oxazole-4-carboxamide

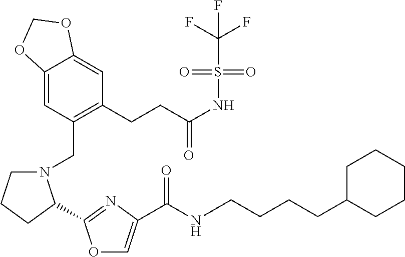

¹H-NMR (CDCl₃, 300 MHz): 8.34 (s, 1H, ═CH), 7.32 (m, 1H, NH), 6.62 (s, 1H, ArH), 6.60 (s, 1H, ArH), 5.88 (s, 2H, —OCH₂O—), 3.86 (d, 1H, J=11.9 Hz, —NCH₂Ar), 3.74 (t, 1H, J=7.7 Hz, NCH-Oxazole), 3.41 (m, 3H, CONHCH₂—+—NCH₂Ar), 3.05 (m, 1H, —CH₂N—), 2.38 (m, 4H, ArCH₂CH₂CO₂H), 2.00 (c, 1H, J=8.6 Hz, —CH₂N—), 2.69-1.85 (m, 4H, —CH₂—CH₂—), 1.71-1.55 (m, 9H, NHCH₂—CH₂—), 1.36 (m, 2H, —CH₂—CH₂—), 1.25-1.19 (m, 6H, —CH₂—CH₂—). ¹⁹F-NMR (CDCl₃, 300 MHz) 5-79.7. LC-MS (m⁺+1) 657.

### Example 12e

### 2-{1-[5-fluoro-2-(3-oxo-3-{[(trifluoromethyl)sulfonyl]amino}propyl)benzyl]pyrrolidin-2-yl}-N-octyl-1,3-oxazole-4-carboxamide

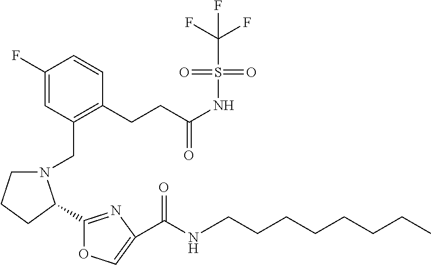

¹H-NMR (CDCl₃, 300 MHz): 8.38 (s, 1H, ═CH), 7.09 (m, 1H, NH), 7.01 (m, 1H, ArH), 6.84 (m, 2H, ArH), 3.92 (d, 1H, J=11.9 Hz, —NCH₂Ar), 3.76 (t, 1H, J=7.7 Hz, NCH-Oxazole), 3.36 (m, 2H, CONHCH₂—), 3.17 (d, 1H, J=11.9 Hz, —NCH₂Ar), 2.98 (m, 1H, —CH₂N—), 2.63-2.30 (m, 5H, ArCH₂CH₂CO₂Me+—CH₂—CH₂-+—CH₂N—), 2.19 (m, 2H, ArCH₂CH₂CO₂Me), 1.92 (m, 2H, —CH₂—CH₂—), 1.25-1.19 (m, 12H, —CH₂—CH₂—), 0.89 (m, 3H, —CH₃). ¹⁹F-NMR (CDCl₃, 300 MHz) δ −79.7, −118.5. LC-MS (M⁺+1) 605.

Examples 12f through 12n are prepared according to General Method 12 by substituting the appropriate reactant to obtain the named compound.

### Example 12f

### 2-{1-(5-methoxy-2-(3-oxo-3-{[(trifluoromethyl)sulfonyl]amino]propyl)benzyl]pyrrolidin-2-yl}-N-octyl-1,3-oxazole-4-carboxamide

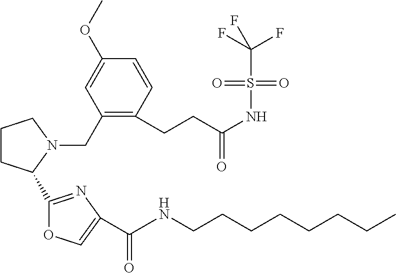

### Example 12g

### 2-{1-(5-methoxy-2-(3-oxo-3-{[(trifluoromethyl)sulfonyl]amino]propyl)benzyl]pyrrolidin-2-yl}-N-pentyl-1,3-oxazole-4-carboxamide

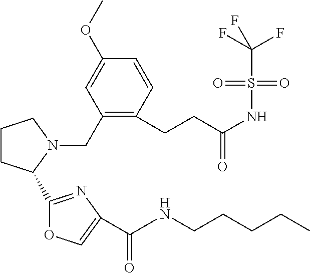

### Example 12h

### 2(S)—N-(4-cyclohexylbutyl)-2-(1-(5-methoxy-2-(3-oxo-3-(fluoropropylsulfonamido)propyl)benzyl)pyrrolidin-2-yl)oxazole-4-carboxamide

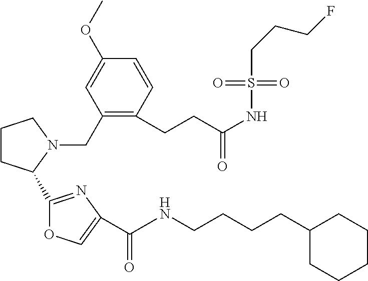

### Example 12i

### (S)—N-(4-cyclohexylbutyl)-2-(1-(5-methoxy-2-(3-oxo-3-(isopropylsulfonamido)propyl)benzyl)pyrrolidin-2-yl)oxazole-4-carboxamide

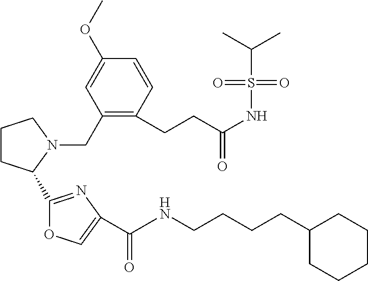

### Example 12j

### (S)—N-(4-cyclohexylbutyl)-2-(1-(5-methoxy-2-(3-oxo-3-(5-chlorothienylsulfonamido)propyl)benzyl)pyrrolidin-2-yl)oxazole-4-carboxamide

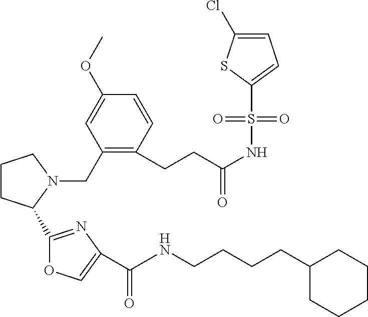

### Example 12k

### (S)—N-(4-cyclohexylbutyl)-2-(1-(5-methoxy-2-(3-oxo-3-(N-diethylsulfamide)propyl)benzyl)pyrrolidin-2-yl)oxazole-4-carboxamide

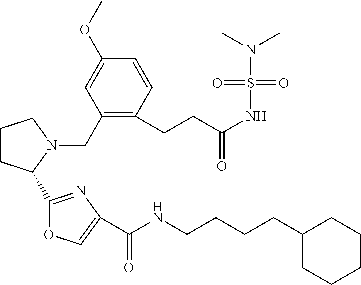

### Example 12l

### (S)—N-(4-cyclohexylbutyl)-2-(1-(5-methoxy-2-(3-oxo-3-(ethylsulfonamido)propyl)benzyl)pyrrolidin-2-yl)oxazole-4-carboxamide

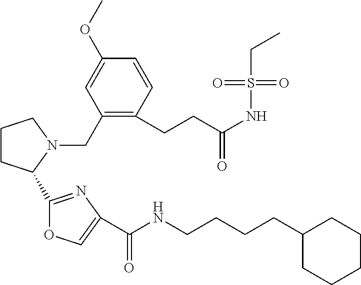

### Example 13

### 2-Bromo-5-methoxy-benzoic acid tert-butyl ester

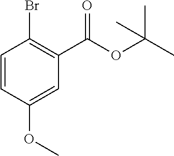

Oxalyl chloride (1.08 mL, 12.33 mmol) and two drops of DMF were added to a solution of 2-bromo-5-methoxy benzoic acid (2.5 g, 10.82 mmol) in toluene (35 mL). The resulting mixture was heated at 50° C. for 1 h.

The reaction was quenched with water (3 mL) and the mixture was extracted with diethyl ether (2×10 mL). The organic layer was dried (MgSO₄), filtered and the solvent was evaporated under vacuum.

Then the reaction was concentrated to dryness under vacuum and the residue was re-dissolved in THF (20 mL). The solution was added to a suspension of potassium tert-butoxide (1.5 g, 13.42 mmol) in THF (30 mL) and the mixture was stirred at room temperature for 16 h,

After that time, water (25 mL) was added followed by a saturated solution of ammonium chloride (25 mL). The mixture was extracted with diethyl ether (50 mL) and the organic layer was washed with Brine, dried (MgSO₄), filtered and the solvent was evaporated under vacuum to isolate the title compound as a colorless solid. (Yield=63%)

¹H-NMR (CDCl₃, 300 MHz): 7.49 (d, 1H, J=8.8 Hz, ArH), 7.24 (d, 1H, J=2.4 Hz, ArH), 6.85 (dd, 1H, J=8.8, 2.4 Hz, ArH), 3.83 (s, 3H, ArOCH₃), 1.63 (s, 9H, CO₂tBu).

### Example 14

### 2-((E)-2-Ethoxycarbonyl-vinyl)-5-methoxy-benzoic acid tert-butyl ester

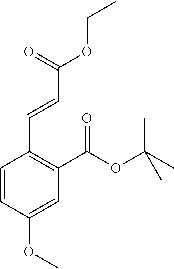

A mixture of Example 21 (1.93 g, 6.74 mmol), ethyl acrylate (1.1 mL, 10.11 mmol), tryethylamine (2.82 mL, 20.22 mmol), tri(o-tolyl)phosphine (0.082 g, 0.27 mmol) and palladium acetate (0.03 g, 0.135 mmol) in toluene (20 mL) was refluxed for 18 h.

Then the reaction was concentrated to dryness under vacuum and the residue was partitioned between ethyl acetate (50 mL) and 2M solution of HCl (50 mL). The organic layer was separated, washed with brine, dried (MgSO₄), filtered and the solvent evaporated to isolate the title compound as an oil.

¹H-NMR (CDCl₃, 300 MHz): 8.34 (d, 1H, J=15.7 Hz, ArCH═CH—CO₂Et), 7.49 (d, 1H, J=8.8 Hz, ArH), 7.24 (d, 1H, J=2.4 Hz, ArH), 6.85 (dd, 1H, J=8.8, 2.4 Hz, ArH), 6.23 (d, 1H, J=15.7 Hz, ArCH═CH—CO₂Et), 4.27 (q, 2H, J=7.5 Hz, —CO₂CH₂CH₃), 3.88 (s, 3H, ArOCH₃), 1.64 (s, 9H, CO₂tBu), 1.35 (t, 3H, J=7.5 Hz, —CO₂CH₂CH₃).

### Example 15

### 2-((E)-2-Ethoxycarbonyl-vinyl)-5-methoxy-benzoic acid

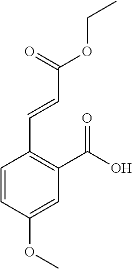

A solution of Example 22 (6.74 mmol), triethyl silane (5.4 mL, 33.7 mmol and TFA (6.75 mL, 87.62 mmol) in dichloromethane (15 mL) was stirred for 30 min at room temperature and then refluxed for 2.5 h.

Then the reaction was concentrated to dryness under vacuum and the residue was purified by column in a 50G Silica cartridge using a gradient from isohexane/ethyl acetate 3:1 to isohexane/ethyl acetate 1:3 to isolate the title compound as a light brown solid (88%).

¹H-NMR (CDCl₃, 300 MHz): 8.34 (d, 1H, J=15.7 Hz, ArCH═CH—CO₂Et), 7.49 (d, 1H, J=8.8 Hz, ArH), 7.24 (d, 1H, J=2.4 Hz, ArH), 6.85 (dd, 1H, J=8.8, 2.4 Hz, ArH), 6.23 (d, 1H, J=15.7 Hz, ArCH═CH—CO₂Et), 4.27 (q, 2H, J=7.5 Hz, —CO₂CH₂CH₃), 3.88 (s, 3H, ArOCH₃), 1.35 (t, 3H, J=7.5 Hz, —CO₂CH₂CH₃).

### Example 16

### 2-(2-Ethoxycarbonyl-ethyl)-5-methoxy-benzoic acid

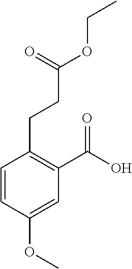

The Example 23 (1.4 g, 5.6 mmol) was dissolved in a mixture of ethanol (20 mL) and dioxane (20 mL). Palladium on carbon catalyst (140 mg) was added and the suspension was stirred for 18 h at room temperature under a hydrogen atmosphere.

The catalyst was removed by filtration through Hyflo and the filtrate was evaporated under vacuum to give a yellow solid (90%).

¹H-NMR (CDCl₃, 300 MHz): 7.57 (d, 1H, J=2.4 Hz, ArH), 7.24 (d, 1H, J=8.8 Hz, ArH), 7.04 (dd, 1H, J=8.8, 2.4 Hz, ArH), 4.13 (q, 2H, J=7.5 Hz, —CO₂CH₂CH₃), 3.85 (s, 3H, ArOCH₃), 3.25 (t, 2H, J=7.5 Hz, ArCH₂CH₂CO₂Et), 2.69 (t, 2H, J=7.5 Hz, ArCH₂CH₂CO₂Et), 1.35 (t, 3H, J=7.5 Hz, —CO₂CH₂CH₃).

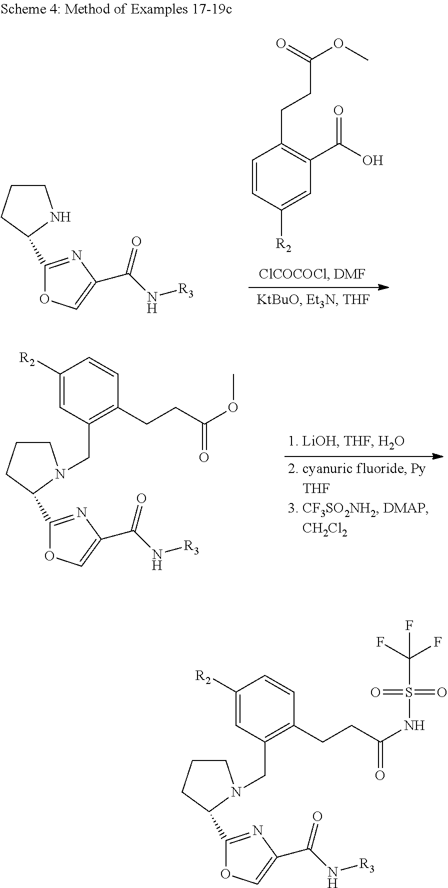

### Example 17

### 3-(2-{(S)-2-[4-(4-Cyclohexyl-butylcarbamoyl)-oxazol-2-yl]-pyrrolidine-1-carbonyl}-4-methoxy-phenyl)-propionic acid ethyl ester

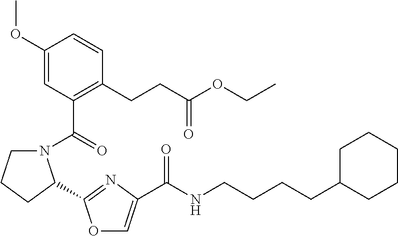

Oxalyl chloride (0.083 mL, 0.95 mmol) and one drop of DMF were added to a solution of Example 24 (0.2 g, 0.79 mmol) in toluene (5 mL). The resulting mixture was heated at 50° C. for 1 h.

The reaction was quenched with water (3 mL) and the mixture was extracted with diethyl ether (2×10 mL). The organic layer was dried (MgSO₄), filtered and the solvent was evaporated under vacuum.

Then the reaction was concentrated to dryness under vacuum and the residue was re-dissolved in THF (2 mL). The solution was added to a solution of (S)-2-Pyrrolidin-2-yl-oxazole-4-carboxylic acid (4-cyclohexyl-butyl)-amide (0.23 g, 0.72 mmol) of potassium tert-butoxide (1.5 g, 13.42 mmol) and triethylamine (0.11 mL, 0.79 mmol) in THF (5 mL) and the mixture was stirred at room temperature for 16 h,

After that time, water (25 mL) was added followed by a saturated solution of ammonium chloride (25 mL). The mixture was extracted with diethyl ether (50 mL) and the organic layer was washed with Brine, dried (MgSO₄), filtered and the solvent was evaporated under vacuum.

The residue was purified by column in a 20G Silica cartridge using a gradient from iso-hexane/ethyl acetate 3:1 to iso-hexane/ethyl acetate 1:5 to isolate the title compound as a colorless solid (50%).

¹H-NMR (CDCl₃, 300 MHz): 8.13 (s, 1H, ═CH), 7.19 (d, 1H, J=8.4 Hz, ArH), 7.05 (bs, 1H, NH), 6.88 (dd, 1H, J=2.4, 8.4 Hz, ArH), 6.79 (d, 1H, J=2.4 Hz, ArH), 4.11 (q, 2H, J=7.5 Hz, —CO₂CH₂CH₃), 3.89 (t, 1H, J=7.7 Hz, NCH-oxazole), 3.80 (s, 3H, Ar—OCH₃), 3.37 (m, 4H, CONHCH₂—+ArCH₂CH₂CO₂Et), 2.90 (m, 2H, —ArCONCH₂—), 2.59 (m, 2H, ArCH₂CH₂CO₂Me), 2.36 (m, 1H, —CH₂—CH₂—), 2.11 (m, 3H, —CH₂—CH₂—), 1.71-1.55 (m, 8H, NHCH₂—CH₂—), 1.36 (m, 6H, —CH₂—CH₂—), 1.22 (m, 3H, —CO₂CH₂CH₃), 0.89 (m, 3H, —CH₂—CH₂—).

### Example 18

### 3-(2-{(S)-2-[4-(4-Cyclohexyl-butylcarbamoyl)-oxazol-2-yl]-pyrrolidine-1-carbonyl}-4-methoxy-phenyl)-propionic acid

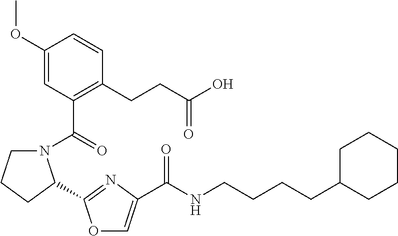

The ester (Example 25) (0.145 g, 0.26 mmol) was dissolved in THF (3 mL) and a solution of NaOH (0.042 g, 1.05 mmol) in water (1 mL) was added. The resulting mixture was stirred at room temperature for 16 h.

Then, EtOAc was added (10 mL) and the solution was neutralized with a 2M solution of HCl. The organic layer was separated, washed with brine (10 mL) and dried (Na₂SO₄). The mixture was filtered and the solvent was evaporated to give crude product.

The compound was purified by column chromatography on a 10 g SPE cartridge, using as eluent: 2% MeOH/98% CH₂Cl₂, to give the carboxylic acid as a white solid (80%).

The residue was purified by column in a 20G Silica cartridge using a gradient from ethyl acetate to ethyl acetate/methanol 9:1 to isolate the title compound as a colorless solid (70%).

¹H-NMR (CDCl₃, 300 MHz): 8.20 (s, 1H, ═CH), 7.19 (d, 1H, J=8.4 Hz, ArH), 7.08 (bs, 1H, NH), 6.87 (dd, 1H, J=2.4, 8.4 Hz, ArH), 6.79 (d, 1H, J=2.4 Hz, ArH), 3.89 (t, 1H, J=7.7 Hz, NCH-oxazole), 3.80 (s, 3H, Ar—OCH₃), 3.37 (m, 4H, CONHCH₂—+ArCH₂CH₂CO₂H), 2.90 (m, 2H, —ArCONCH₂—), 2.59 (m, 2H, ArCH₂CH₂CO₂H), 2.36 (m, 1H, —CH₂—CH₂—), 2.11 (m, 3H, —CH₂—CH₂—), 1.71-1.55 (m, 8H, NHCH₂—CH₂—), 1.36 (m, 6H, —CH₂—CH₂—), 0.89 (m, 3H, —CH₂—CH₂—).

### General Method 19

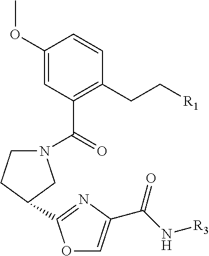

To a solution of saturated acid (Example 26) (0.16 mmol) in THF (10 mL), under nitrogen atmosphere, was added pyridine (1.2 mmol) and cyanuric fluoride (1.2 mmol) and the resulting mixture was refluxed for 4 h. The reaction mixture was left to cool to room temperature, then diluted with ethyl acetate (15 mL) and water (10 mL). The organic layer was separated, washed with a saturated solution of NaHCO₃ (10 mL), then saturated brine (10 mL), dried (MgSO₄), filtered and the solvent was evaporated under vacuum.

The crude product was re-dissolved in CH₂Cl₂ (10 mL) and DMAP (0.64 mmol) and alkylsulfonamide (0.64 mmol) were added. The resulting mixture was stirred at room temperature under nitrogen for 16 h.

After this time, the reaction mixture was diluted with more CH₂Cl₂ (15 mL) and water (10 mL) was added. The organic layer was separated, washed with a 2M solution of HCl (5 mL), then saturated brine (10 mL) and dried (MgSO₄), filtered and the solvent was evaporated under vacuum.

The residue was purified by column chromatography through a 10 g SPE silica cartridge using a solvent gradient starting from ethyl acetate to ethyl acetate/methanol 9:1, to isolate the title compound as thick oil (60%).

### Example 19a

### 2-{(S)-1-[2-(3-Ethanesulfonylamino-3-oxo-propyl)-5-methoxy-benzoyl]-pyrrolidin-2-yl}-oxazole-4-carboxylic acid (4-cyclohexyl-butyl)-amide

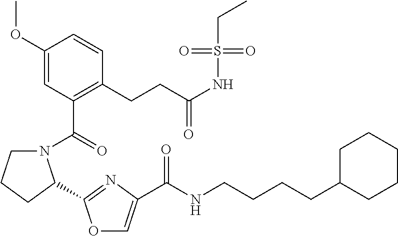

¹H-NMR (CDCl₃, 300 MHz): 8.16 (s, 1H, ═CH), 7.17 (d, 1H, J=8.4 Hz, ArH), 6.90 (m, 2H, ArH+CONH), 6.79 (d, 1H, J=2.4 Hz, ArH), 3.82 (s, 3H, Ar—OCH₃), 3.37 (m, 4H, —NHSO₂CH₂CH₃+ArCH₂CH₂CONHSO₂), 3.24 (m, 3H, NCH-oxazole+CONHCH₂—), 2.90 (m, 2H, —ArCONCH₂—), 2.59 (m, 2H, ArCH₂CH₂CONHSO₂), 2.36 (m, 1H, —CH₂—CH₂—), 2.11 (m, 3H, —CH₂—CH₂—), 1.71-1.55 (m, 8H, NHCH₂—CH₂—), 1.36 (m, 6H, —CH₂—CH₂—), 1.19 (m, 3H, NHSO₂CH₂CH₃), 0.89 (m, 3H, —CH₂—CH₂—). LC-MS (M⁺+1) 617.

### Example 19b

### 2-{(S)-1-[2-(3-Methanesulfonylamino-3-oxo-propyl)-5-methoxy-benzoyl]-pyrrolidin-2-yl}-oxazole-4-carboxylic acid (4-cyclohexyl-butyl)-amide

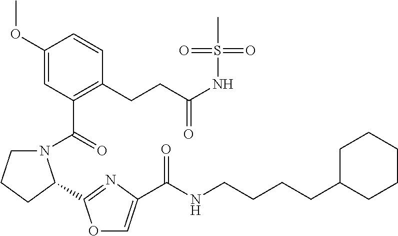

¹H-NMR (CDCl₃, 300 MHz): 8.16 (s, 1H, ═CH), 7.17 (d, 1H, J=8.4 Hz, ArH), 6.90 (m, 2H, ArH+CONH), 6.79 (d, 1H, J=2.4 Hz, ArH), 3.82 (s, 3H, Ar—OCH₃), 3.44 (m, 5H, NCH-oxazole+CONHCH₂+ArCH₂CH₂CONHSO₂—), 3.02 (s, 3H, —NHSO₂CH₃), 2.90 (m, 2H, —ArCONCH₂—), 2.59 (m, 2H, ArCH₂CH₂CONHSO₂), 2.36 (m, 1H, —CH₂—CH₂—), 2.11 (m, 3H, —CH₂—CH₂—), 1.71-1.55 (m, 8H, NHCH₂—CH₂—), 1.36 (m, 6H, —CH₂—CH₂—), 0.89 (m, 3H, —CH₂—CH₂—). LC-MS (M⁺+1) 603.

### Example 19c

### 2-{(S)-1-[2-(3-Trifluoromethanesulfonylamino-3-oxo-propyl)-5-methoxy-benzoyl]-pyrrolidin-2-yl}-oxazole-4-carboxylic acid (4-cyclohexyl-butyl)-amide

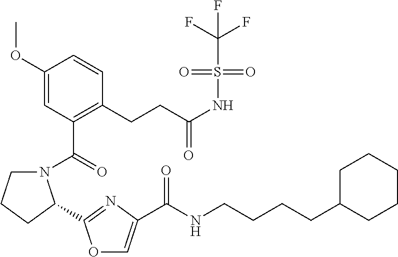

¹H-NMR (CDCl₃, 300 MHz): 8.18 (s, 1H, ═CH), 7.17 (d, 1H, J=8.4 Hz, ArH), 6.88 (m, 2H, ArH+CONH), 6.74 (d, 1H, J=2.4 Hz, ArH), 3.77 (s, 3H, Ar—OCH₃), 3.36 (m, 5H, NCH-oxazole+CONHCH₂+ArCH₂CH₂CONHSO₂—), 2.74 (m, 2H, —ArCONCH₂—), 2.35 (m, 3H, ArCH₂CH₂CONHSO₂+—CH₂—CH₂—), 2.11 (m, 3H, —CH₂—CH₂—), 1.71-1.55 (m, 8H, NHCH₂—CH₂—), 1.36 (m, 6H, —CH₂—CH₂—), 0.89 (m, 3H, —CH₂—CH₂—).

¹⁹F-NMR (CDCl₃, 300 MHz) δ −79.3

LC-MS (M⁺+1) 657

The compounds of Examples 12, and 19 are tested for FAAH inhibitory activity as follows:

Method 1: Membranes obtained from rat brain are incubated with 2 mM [¹⁴C]-AEA, 30 min at 37° C. at pH values ranging from 9.00 to 10.00 in presence and absence of tested compounds in a final volume of 500 mL. Incubation is stopped by extraction with CHCl₃/MeOH (1:1) and the aqueous phases containing [¹⁴C]-Ethanolamine produced by [¹⁴C]-AEA hydrolysis are measured.

Method 2: 2 mg/sample of human FAAH recombinant are incubated with 2 mM of [¹⁴C]-AEA for 30 min at 37° C. at pH values ranging from 9.00 to 10.00 in presence and absence of compounds. The final volume of incubation is maintained less than 0.2 mL in order to facilitate enzyme-substrate complex formation. The incubation is stopped by extraction with CHCl₃/MeOH (1:1) and the aqueous phases containing [14C]-Ethanolamine produced by [14C]-AEA hydrolysis is measured.

The results of the testing are reported in the Tables, below.

TABLE 1
Prolines acylsulfonamides as FAAH inhibitors

| EXAM- | | | | | | | | | |
|---|---|---|---|---|---|---|---|---|---|
| PLE No | FAAH | FP | DP | EP1 | EP2 | EP3 | EP4 | IP | TP |
| 12a | 1000 | 350 | <1 | 50 | 6400 | 80 | 40 | 550 | <1 |
| 12b | 4500 | 460 | <1 | 70 | 5500 | 100 | 40 | 9500 | <1 |
| 12c | 200 | 270 | 17 | 20 | 830 | 47 | 10 | 860 | <1 |
| 12d | 740 | 360 | 3 | 60 | 3900 | 150 | 7 | 1000 | 0.3 |
| 12e | 2100 | 270 | 20 | 20 | 2500 | 110 | 20 | 290 | <1 |
| 12f | 500 | 380 | <1 | 60 | 1100 | 140 | 20 | 400 | <1 |

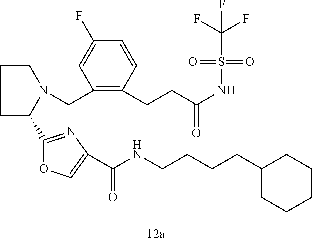

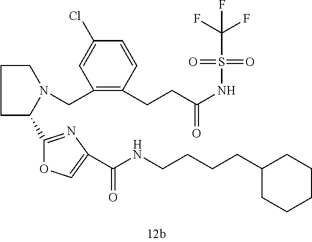

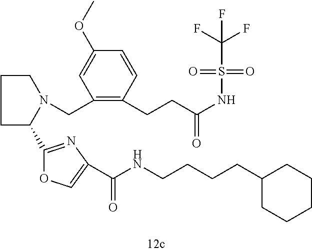

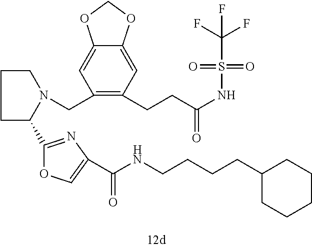

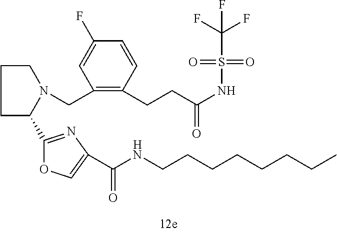

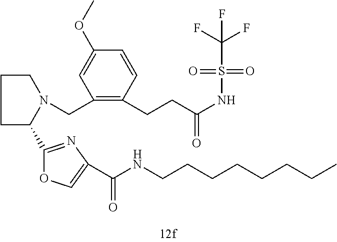

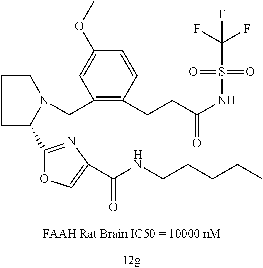

FAAH Rat brain IC₅₀ (nM)
(FLIPR) K_{b} (nM), NA = inactive

The following conclusions may be drawn from the data reported in Table 1:

An alkoxy group for R₂ may be preferred.

Unsaturation in the ethylenyl group linking the acylsulfonamide and the phenyl groups of the molecule may diminish FAAH inhibitor activity.

TABLE 2
Prolines acylsulfonamides as FAAH inhibitors

| EXAM- | | | | | | | | | |
|---|---|---|---|---|---|---|---|---|---|
| PLE No | FAAH | FP | DP | EP1 | EP2 | EP3 | EP4 | IP | TP |
| 12h | 100 | 2000 | 8 | 830 | 7800 | 50 | 150 | NA | 8 |
| 12i | 150 | 1500 | 44 | 1900 | NA | 480 | 210 | NA | 1 |
| 12j | 210 | 2300 | 28 | 240 | 900 | 10 | 360 | NA | 4 |
| 12k | 200 | 1400 | 80 | 3100 | 4000 | 5600 | 400 | 2900 | 13 |
| 12l | 240 | 1600 | 40 | 180 | 1800 | 910 | 160 | 6900 | 11 |

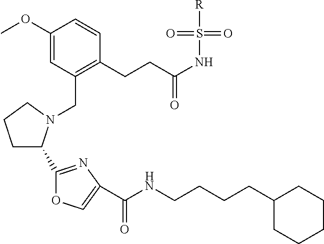

12h, R = CH₂CH₂CH₂F
12i, R = iPr 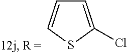
12k, R = N(CH₃)₂
12l, R = Et
FAAH Rat brain IC₅₀ (nM)
(FLIPR) K_{b} (nM), NA = inactive

The following conclusions may be drawn from the data reported in Table 2:

R₃ may preferably a cycloalkyl group, such as an cycloalkyl-n-alkyl group, e.g. cyclohexyl-n-butyl.

TABLE 3
Proline Amides Scaffold

| Exam- | | | | | | | | | |
|---|---|---|---|---|---|---|---|---|---|
| ple No | FAAH | FP | DP | EP1 | EP2 | EP3 | EP4 | IP | TP |
| 19 | 180 | 1900 | 85 | 3220 | 7740 | 650 | 580 | NA | 0.6 |
| 19a | 20 | 1900 | 80 | 300 | 1200 | 600 | 300 | 2000 | 20 |
| 19b | 30 | 2200 | 120 | 2200 | 3000 | 1500 | 430 | NA | 1 |

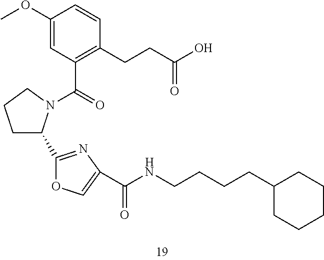

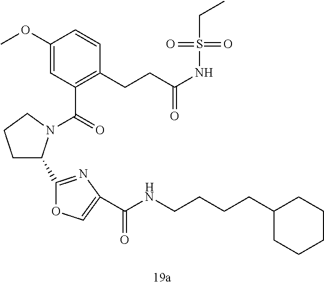

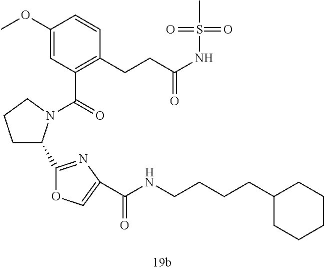

The following conclusions may be drawn from the data reported in Table 3:

R₇ may preferably an alkyl group.

The claims are not to be limited in scope by the exemplified embodiments, which are only intended as illustrations of specific embodiments. Various modifications of the embodiments, in addition to those disclosed herein, will be apparent to those skilled in the art by a careful reading of the specification, including the claims, as originally filed. In particular, while some embodiments have been illustrated by the treatment of pain, the method of using the above compounds to treat any of the diseases and/or conditions of humans that are mediated by FAAH and/or the above described PG receptors, especially conditions that benefit from blocking and antagonizing both the FAAH inhibiting activity and the activity at one or more PG receptors, e.g. the DP₁, FP, EP₁, EP₃, TP, and/or EP₄ receptors, are contemplated by this disclosure. It is intended that all such modifications will fall within the scope of the appended claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

1. A method of treating a patient suffering from pain comprising administering a fatty acid amide inhibiting amount of a compound represented by the formula: 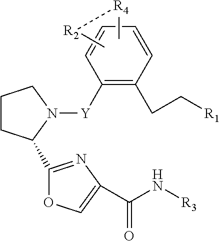
wherein a dashed line indicates the presence or absence of a bond;
R₁ is an acyl sulfonamide moiety;
R₂ and R₄ are independently H, alkyl, halo or alkyloxy;
R₃ is H or alkyl; and
Y is CO or (CH₂)ₙ, wherein n is 1, 2, or 3.

2. The method of claim 1, wherein R₁ is CON(R₇)SO₂R₇, wherein R₇ is H, heteroaryl, dialkylamino, hydrocarbyl, or substituted hydrocarbyl.

3. The method of claim 2, wherein R₇ is alkyl, dialkylamino, heteroaryl, or haloalkyl.

4. The method of claim 1, wherein R₁ is CON(H)SO₂R₇.

5. The method of claim 4, wherein R₇ is methyl, ethyl, i-propyl, fluoropropyl, trifluoromethyl, chlorothienyl, or dimethylamino.

6. The method of claim 1, wherein R₂ is F, Cl, OCH₃, or R₂ . . . R₄ is O(CH₂)O.

7. The method of claim 3, wherein R₃ is alkyl.

8. The method of claim 7, wherein R₃ is (CH₂)ₙCH₂R₅, wherein n is 4, 5, 6, 7, 8, or 9, and R₅ is H or cyclohexyl.

9. The method of claim 8, wherein R₅ is a cyclohexyl moiety.

10. The method of claim 9, wherein R₂ is OCH₃.

11. The method of claim 1, wherein the compound is:

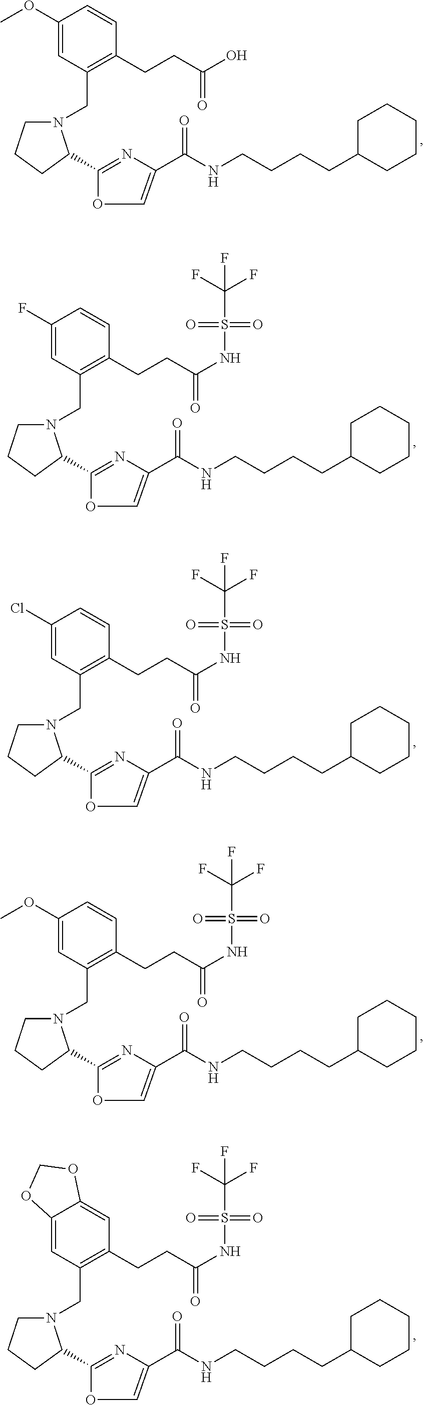

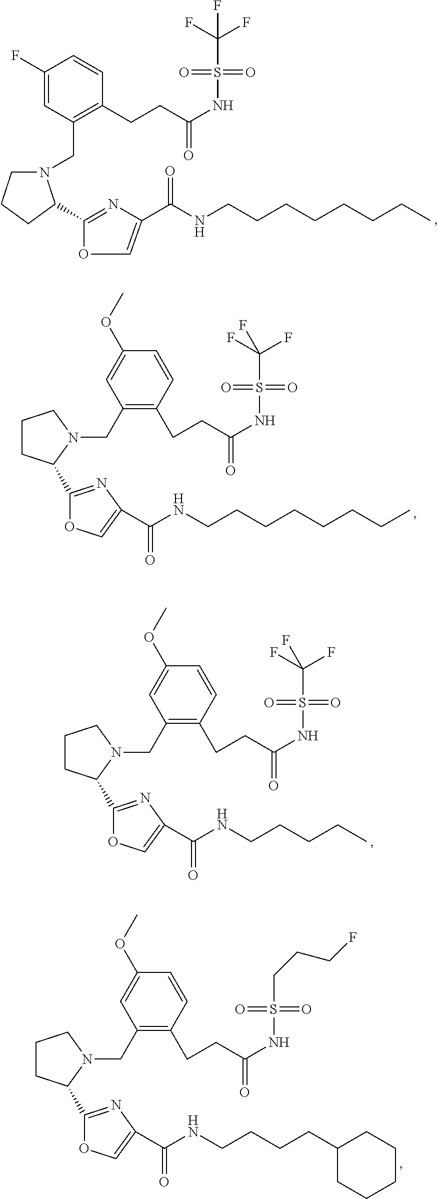

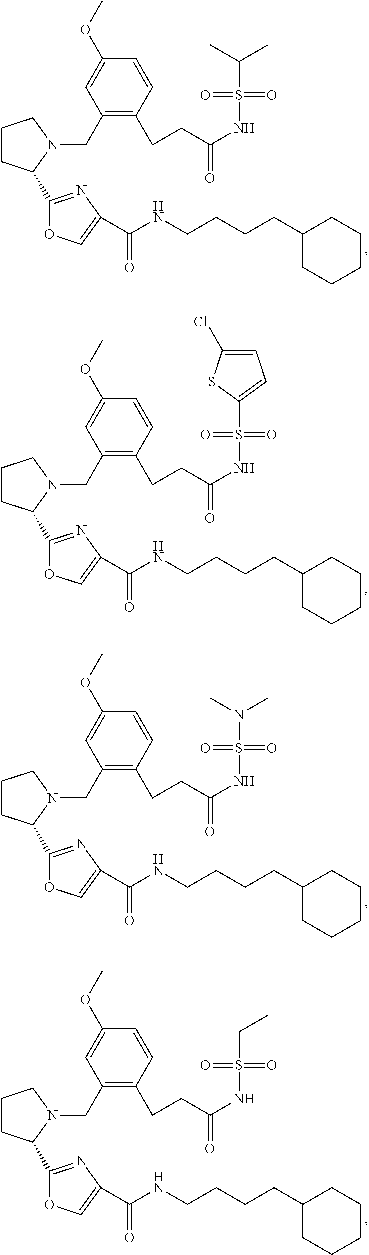

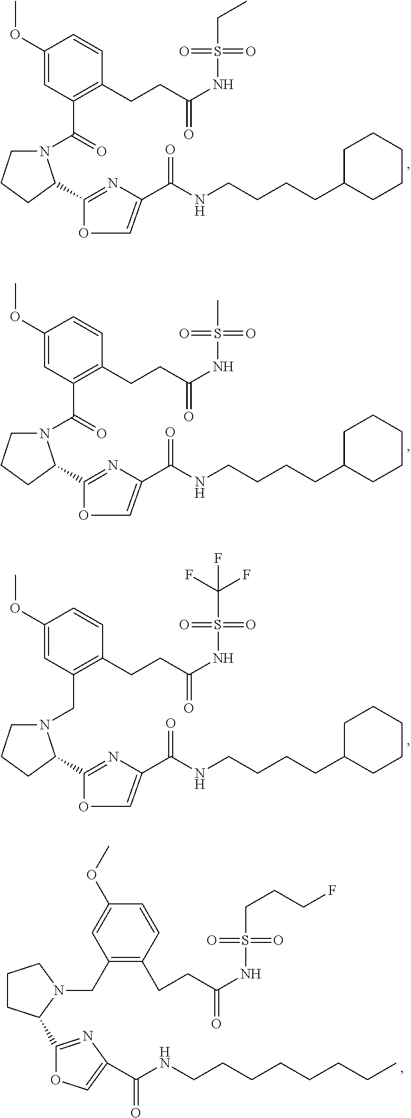

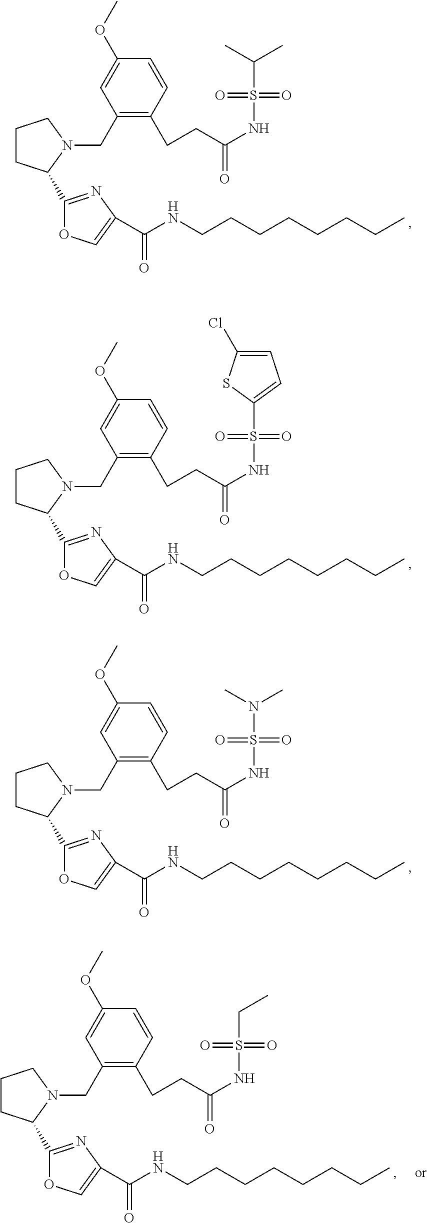

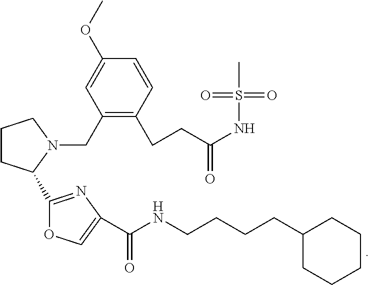

12. A method of treating a patient having a condition mediated by FAAH which comprises administering a fatty acid amide inhibiting amount of a compound represented by the formula: 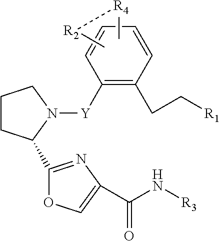
wherein a dashed line indicates the presence or absence of a bond;
R₁ is an acyl sulfonamide moiety;
R₂ and R₄ are independently H, alkyl, halo or alkyloxy;
R₃ is H or alkyl; and
Y is CO or (CH₂)ₙ, wherein n is 1, 2, or 3.

13. The method of claim 12, wherein the condition is a pain-related condition.

14. The method of claim 12, wherein the condition is mediated by FAAH and at least one PG receptor.

15. A method of claim 14, wherein the condition is mediated by at least two PG receptors.

16. The method of claim 12, wherein the compound has both fatty acid amide hydrolase inhibiting activity and antagonist activity at a PG receptor.

17. A method of treating a patient suffering from pain by administering a fatty acid amide inhibiting amount of a compound, that is an N-alkyl-2-(1-(-2-(3-oxo-3-(hydrocarbyl or substituted hydrocarbylsulfonamido)propyl)benzyl)pyrrolidin-2-yl)oxazole-4-carboxamide.

18. A compound represented by the formula: 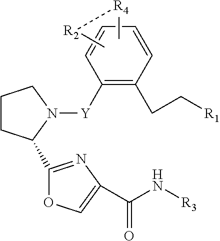
wherein a dashed line indicates the presence or absence of a bond;
R₁ is an acyl sulfonamide moiety;
R₂ and R₄ are independently H, alkyl, halo or alkyloxy;
R₃ is H or alkyl; and
Y is CO or (CH₂)ₙ, wherein n is 1, 2, or 3.

19. The compound of claim 18, wherein R₃ is —(CH₂)₄-cyclohexyl, n-butyl, n-pentyl, n-hexyl, n-heptyl, or n-octyl.

20. The compound of claim 18, wherein the compound is: 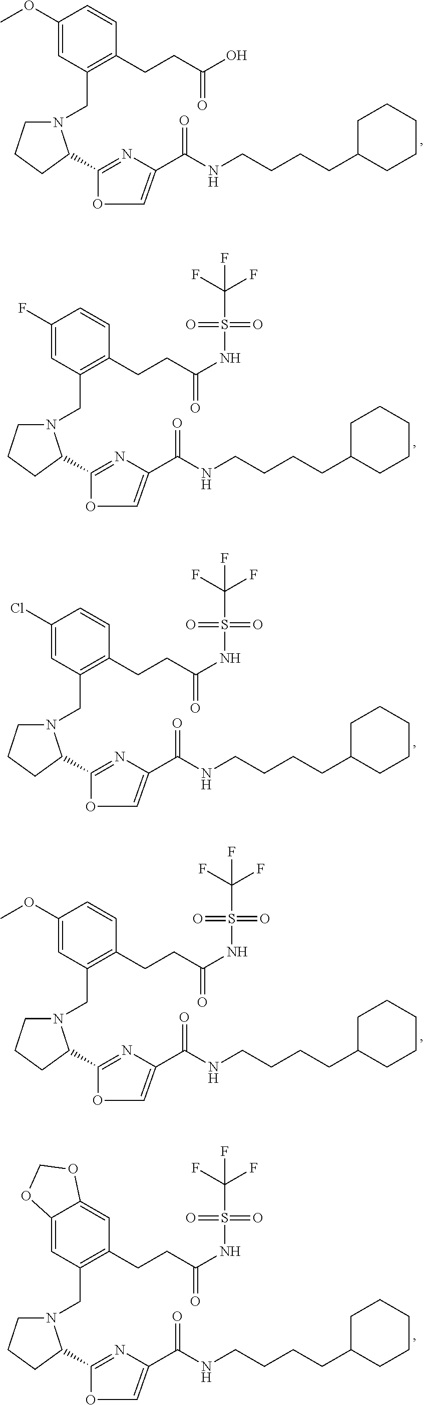 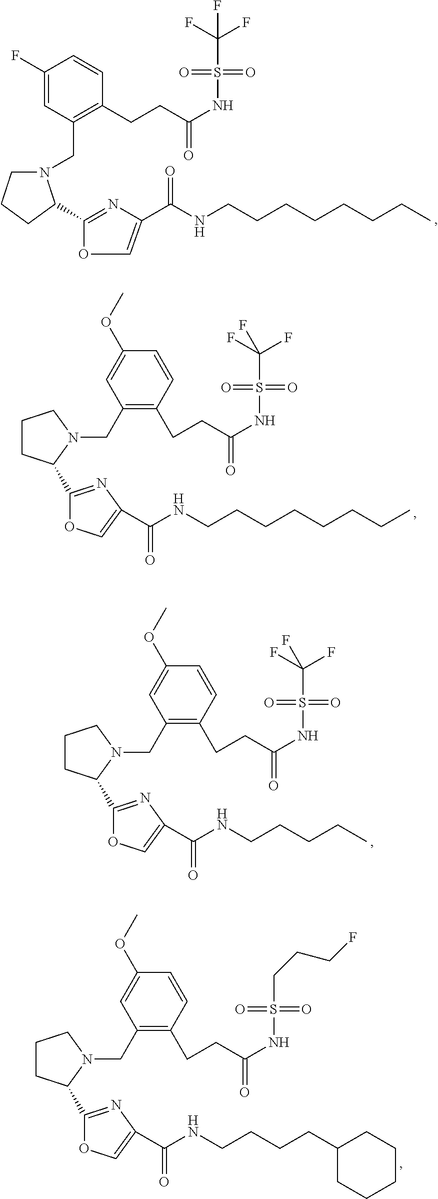 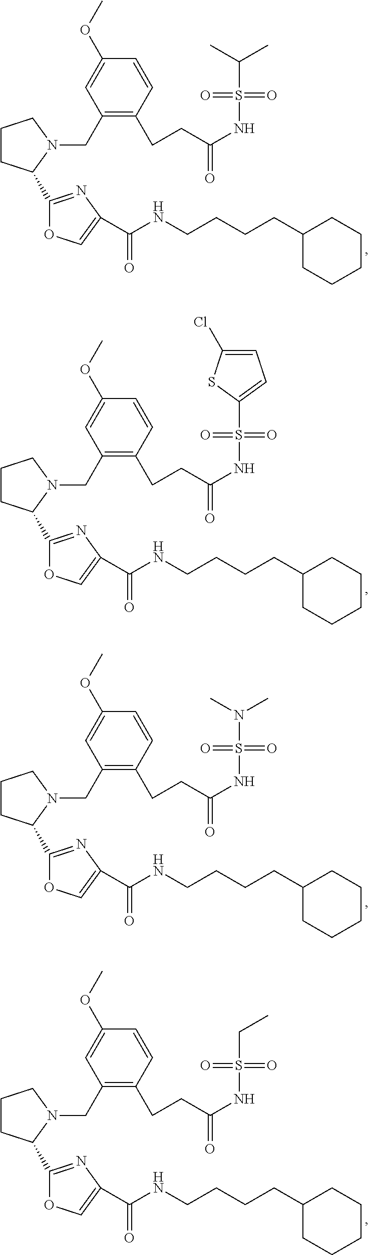 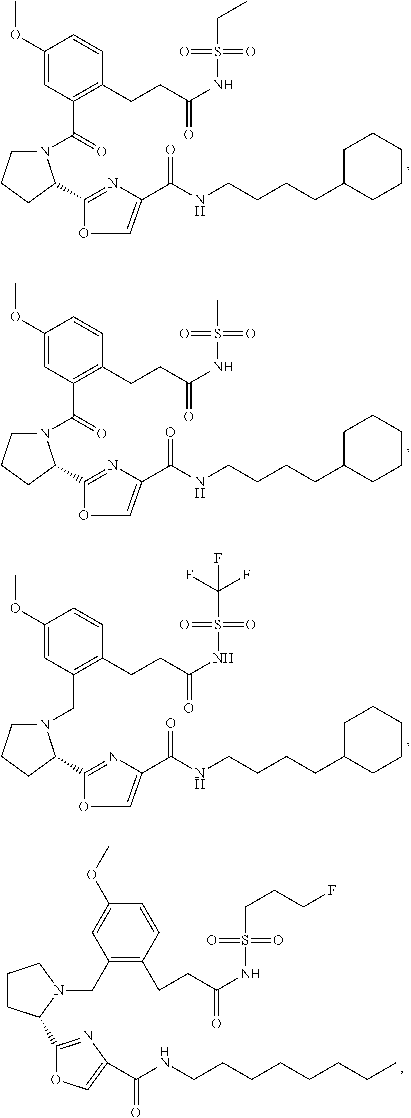 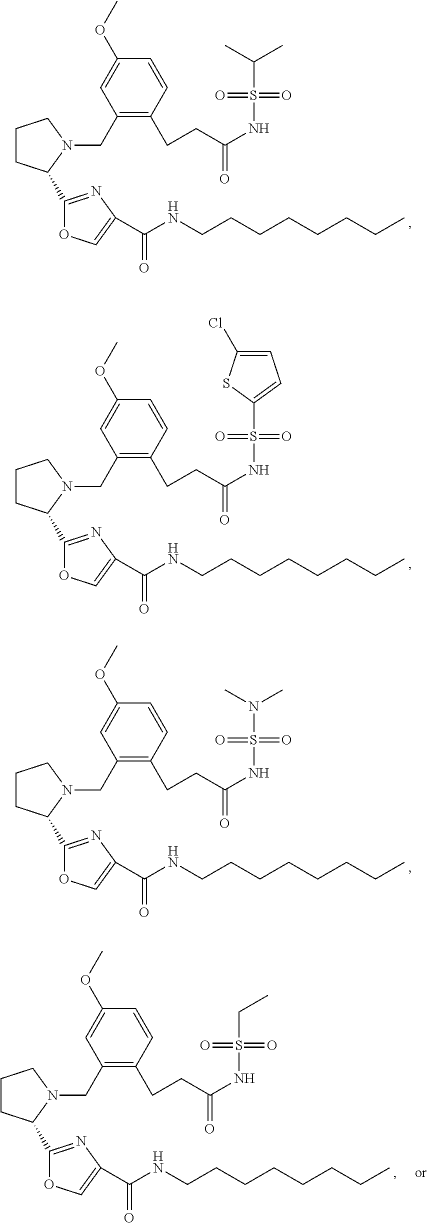 [chemical structure]
